SEQUENCE LISTING (20 sequences)

SEQ ID NO: 1 - protein, 7 aa, Artificial Sequence
  Synthetic peptide
PKKKRKV

SEQ ID NO: 2 - protein, 7 aa, Artificial Sequence
  Synthetic peptide
PKKTRKV

SEQ ID NO: 3 - protein, 2 aa, Artificial Sequence
  Synthetic peptide
GG

SEQ ID NO: 4 - protein, 5 aa, Artificial Sequence
  Synthetic peptide
PLGVR

SEQ ID NO: 5 - protein, 5 aa, Artificial Sequence
  Synthetic peptide
PLGVA

SEQ ID NO: 6 - protein, 2 aa, Artificial Sequence
  Synthetic peptide
KK

SEQ ID NO: 7 - protein, 2 aa, Artificial Sequence
  Synthetic peptide
RR

SEQ ID NO: 8 - protein, 8 aa, Artificial Sequence
  Synthetic peptide
GGPRGLPG

SEQ ID NO: 9 - protein, 6 aa, Artificial Sequence
  Synthetic peptide
HSSKLQ

SEQ ID NO: 10 - protein, 17 aa, Artificial Sequence
  Synthetic peptide
AEAGALVNASSAAHVDV

SEQ ID NO: 11 - protein, 8 aa, Artificial Sequence
  Synthetic peptide
SQNYPIVQ

SEQ ID NO: 12 - protein, 10 aa, Artificial Sequence
  Synthetic peptide
GVVNASCRLA

SEQ ID NO: 13 - protein, 5 aa, Artificial sequence
  Synthetic peptide
PLGLA

SEQ ID NO: 14 - protein, 8 aa, Artificial Sequence
  Synthetic peptide
PKKKRKVK

SEQ ID NO: 15 - protein, 8 aa, Artificial Sequence
  Synthetic peptide
PKKTRKVK

SEQ ID NO: 16 - protein, 6 aa, Artificial Sequence
  Synthetic peptide
PLGLRK

SEQ ID NO: 17 - protein, 6 aa, Artificial Sequence
  Synthetic peptide
PLGVAK

SEQ ID NO: 18 - protein, 11 aa, Artificial Sequence
  Synthetic peptide
PKKKRKVKGGK

SEQ ID NO: 19 - protein, 11 aa, Artificial Sequence
  Synthetic peptide
PKKTRKVKGGK

SEQ ID NO: 20 - protein, 17 aa, Artificial Sequence
  Synthetic peptide
PKKKRKVKGGKPLGVRK

(19) United States
(12) Patent Application Publication
(10) Pub. No.: US 2009/0203877 A1
(43) Pub. Date: Aug. 13, 2009

(54) X-RAY-DENSE CONJUGATE

(75) Inventors: Stefan Heckl, Tuebingen (DE); Alexander Sturzu, Tuebingen (DE)

Correspondence Address: KLARQUIST SPARKMAN, LLP, 121 SW SALMON STREET, SUITE 1600, PORTLAND, OR, 97204, US

(73) Assignee: Eberhard-Karls-Universitaet Tuebingen Universitaetsklinkum

(21) Appl. No.: 12/360,035

(22) Filed: Jan. 26, 2009

Related U.S. Application Data
(63) Continuation of application No. PCT/EP2007/006678, filed on Jul. 27, 2007.

(30) Foreign Application Priority Data
Jul. 27, 2006 (DE) DE10 2006035577.6

(51) Int. Cl. C07K 7/08; C07K 2/00; C07K 7/06

(52) U.S. Cl. 530/322; 530/300; 530/331; 530/330; 530/329; 530/328; 530/327; 530/326

(57) ABSTRACT

The present invention relates to an X-ray dense conjugate, the use of the conjugate for producing a diagnostic and therapeutic composition, a pharmaceutical and/or diagnostic composition, which comprises said conjugate, a method for the diagnostic and/or analytical treatment of biological material or a living being, and a method for the therapeutic treatment of a living being.

### CROSS-REFERENCE TO RELATED APPLICATIONS

This application is a continuation of copending International Patent Application PCT/EP 2007/006678 filed on Jul. 27, 2007 and designating the United States, which was not published under PCT Article 21 (2) in English, and claims priority of German Patent Application DE 10 2006 035 577.6 filed on Jul. 27, 2006, which is incorporated herein by reference.

### BACKGROUND OF THE INVENTION

1. Field of the Invention

The present invention relates to an X-ray-dense conjugate, the use of said conjugate for the production of a diagnostic and therapeutic composition, a pharmaceutical and/or a diagnostic composition which comprises said conjugate, a method for the diagnostic and/or analytic treatment of biological material or a living being, as well as a method for the therapeutic treatment of a living being.

2. Related Prior Art

The computer tomography (CT) is a very prevalent imaging method in emergency and routine investigations which, in comparison to magnetic resonance tomography, enables a particular high resolution, e.g. of the parenchyma of the lung.

To increase the contrast in the CT contrast media are used. At present mainly substances containing iodine are used, such as e.g. Iopromid (Ultravist®) or Iobitridol (Xenetix®). These contrast media comprise as a skeleton structure due to its X-ray-dense iodine atoms 2,3,5-triiodobenzoic acid (TIBA) which primarily was mainly

used to induce a premature anthesis or time of ripening of plants; Galston A. W. The Effect of 2,3,5-Triiodobenzoic Acid on the Growth and Flowering of Soybeans. American Journal of Botany 34, 356-360 (1947). Since TIBA does not dissolve in water these contrast media comprise multiple substitutions where e.g. a plurality of OH-groups are coupled to TIBA. By this way the mentioned contrast media obtain a high molecular weight, namely of 791 Da in the case of Iopromid and of 835 Da in the case of Iobitridol.

The currently used contrast media containing iodine have the disadvantage that they cannot penetrate the membrane of the biological cells and therefore only enrich in the space between the cells, i.e. in the interstitial space of tumors (cf. Krause W., Delivery of diagnostic agents in computed tomography, Adv. Drug Deliv. Rev., 159-173 (1999)).

In the document U.S. Pat. No. 5,567,410 and the publication of Torchilin V. P., Polymeric contrast agents for medical imaging, Current Pharmaceutical Biotechnology, 183-215 (2000), a contrast medium is proposed which comprises an amphiphilic character and form micelles in physiological liquids. The contrast medium contains three molecules of TIBA per entire molecule, which are bound to a carboxylic acid backbone. Both components create a hydrophobic block. This hydrophobic block is in turn bound to a hydrophilic polymeric compound which consists of monomethoxypolyethylenglycol (MPEG). Such MPEG compound is very large and has a molecular weight of approximately 12 kDa resulting in a weight of the whole molecule of the known contrast medium of up to 30 kDa. Due to the enormous size as well as the amphiphilic nature of the whole molecule the latter cannot exit the blood stream and can therefore not infiltrate the interstitial space or, much less, the surrounding tissue. For this reason the authors propose to use the contrast medium due to its continuance in the blood stream exclusively for the imaging of blood streams as a so-called “blood pool” contrast medium, e.g. within the context of the angiography, which is eliminated from the body after a short time.

A comparable “blood pool” contrast medium having a very large molecular weight is described in the publication of Fu et al., Dendritic ionidated contrast agents with PEG-cores for CT imaging: synthesis and preliminary characterization, Bioconjugate Chem. 17, 1043-1056 (2006), which comprises triiodophtalamide groups which are coupled via beta-alanine to polylysine in the form of “lysine saplings”.

From the US 2005/0119470 A1 a composition is known which consists of an iodine containing compound, namely 2,3,5-triiodobenzoic acid (TIBA) and two peptides or nucleic acids, which can at least partially hybridize to each other. This composition is not proposed as a contrast medium but as “RNA silencer”.

Since the known extracellular contrast media are not able to infiltrate biological cells, e.g. the tissue of a tumor cannot exactly be differentiated from healthy tissue. The boundaries of a tumor are merely displayed in a blurred manner. If the mentioned extracellular contrast media are administered during or directly after an operation the contrast media runs along the space between the cells, opened by the surgeon, beyond the boundaries of the tumor.

In the WO 2006/069677 A2 a composition is described which comprises a derivative of 2,3,5-triiodobenzoic acid (TIBA) and a “targeting group”, e.g. a nuclear localization sequence (NLS), in combination with a material for the production of an implantable medical device and a therapeutically active agent.

If a mamma carcinoma or a brain tumor is stereotactically analyzed by biopsy, at a later stage the position of the biopsy and the obtained histological result have to be unambiguously localized. For this reason currently this location is marked with a metal clip which must be clearly identifiable in CT or magnetic resonance tomography (MRT) controls to be performed at a later stage, but also in a renewed biopsy or operation. However, with this approach many patients have the impression of a foreign body due to the presence of the metal clip in the mammary or the brain. In controls by means of magnetic resonance tomography the metal clips cause susceptibility artifacts and worsen the anatomical resolution of the surrounding tissue; Matsuura H. et al., Quantification of susceptibility artifacts produced on high-field magnetic resonance images by various biomaterials used for neurosurgical implants. Technical note. J. Neurosurg. 97, 1472-5 (2002).

Since the biopsied tumor does not remain of the same size but changes with regard to the size also the metal clip does not remain at the initial side but is displaced. In biopsies in the tissue of the mammary gland after one year the metal clips could be found remote from the initial side of the biopsy, even in another mammary quadrant; Philpotts L. E. & Lee C. H., Clip migration after 11-gauge vacuum-assisted stereotactic biopsy: case report. Radiology 222, 794-796 (2002). If bleeding occur during a biopsy the metal clip can also be flushed out of the area of the biopsy via the puncture channel up to an area under the skin; Parikh J., Ultrasound demonstration of clip migration to Skin within 6 weeks of 11-gauge vacuum-assisted stereotactic breast biopsy. Breast J. 10, 539-542 (2004).

The object of the so-called radiotherapy in the oncology is the entire and targeted elimination of the tumor cells by the use of radioactivity. Here radioactive substances can be used, such as radioactive iodine. At present this radioiodine therapy in human only succeeds for the thyroid carcinoma since this specific kind of tumor expresses the sodium-iodine symporter at the surface of the cells. If radioactive iodine is administered to a patient it is uptaken into the cells together with sodium, i.e. as sodium-iodine symport, and can there develop its radiochemotherapeutical effect.

Since the sodium-iodine symporter is only existing in the thyroid carcinoma other aggressive kinds of cancer, such as brain, prostate or intestine tumors, in humans so far cannot be therapeutically treated by radioactively labelled iodine (¹³¹I), since such tumors cannot uptake the iodine. In order to make e.g. cells of human colon carcinoma accessible to radioactively labelled iodine they have to be transfected with the gene of the sodium-iodine symporter; Scholz I. V. et al., Radioiodine therapy of colon cancer following tissue-specific sodium iodide symporter gene transfer. Gene Ther. 12, 272-80 (2005). These cells of human colon carcinoma then express, similar to thyroid carcinoma cells, the sodium iodine symporter at the cell surface and can then uptake the radioactively labelled iodine.

In the radioiodine therapy of the thyroid carcinoma also healthy tissues, such as the salivary glands, gastric mucosa and the lactating breast, take up the radioactively labelled iodine since there the sodium-iodine symporter is also expressed; Spitzweg C. et al., Analysis of human sodium iodide symporter gene expression in extrathyroidal tissues and cloning of its complementary deoxyribonucleic acids from salivary gland, mammary gland, and gastric mucosa. J. Clin. Endocrinol. Metab. 83, 1746-51 (1998). For this reason with the currently used radioiodine therapy e.g. an inflammation of the salivary glands or a modification of the taste can occur; Alexander C. et al.; Intermediate and long-term side effects of high-dose radioiodine therapy for thyroid carcinoma. Journal of Nuclear Medicine 39, 1551-1554 (1998). During the therapy the patients are housed in specifically shielded rooms since, as a radiation source, they have to be insulated from the environment. The effect does not immediately start.

### SUMMARY OF THE INVENTION

Against this background an object underlying the invention is to provide an X-ray-dense substance which is qualified as a contrast medium, and by which the disadvantages of the currently used iodine contrast media can be avoided. In particular, such a contrast medium should be provided, by means of which in the computer tomography sharper tumor boundaries can be imaged as this is the case with the currently used contrast media.

Another object underlying the invention is to provide a substance, by means of which an improved labelling of a biopsy site is enabled in comparison with the currently used metal clips.

Further, an object underlying to the invention is to provide an X-ray-dense substance which in particular can be therapeutically used for the treatment of tumors, and by which the disadvantages of the current radioiodine therapy can be avoided. In particular, such a substance should be provided which, once administered, rapidly starts to have an effect and which is preferably not radioactive.

These objects are solved by the provision of a conjugate which comprises a first compound having the following formula:

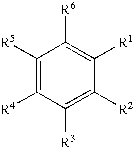

, where R¹, R², R³, R⁴ and R⁵, independently from each other, each correspond to a halogen or a hydrogen, where R⁶ corresponds to a carboxyl group (COOH) or to a isothiocyanate (S═C═N), and comprises at least a first peptide, whereby the conjugate is designed in such a manner that it is cell membrane and nuclear membrane penetrative.

The first compound can comprise 1, 2, 3, 4 or 5 halogen atoms or 1, 2, 3, 4 or 5 hydrogen atoms. The halogens or hydrogens can be arranged at each of the indicated positions R¹ to R⁵. The first compound can comprise different or identical halogens. In accordance with the identity or arrangement of the halogens and in case where R⁶ is a carboxyl group, one refers to e.g. 5-iodobenzoic acid, 4-iodobenzoic acid (5-IBA/MIBA, 4-IBA/MIBA; one iodine atom at positions R¹ or R⁵ or R⁴, respectively), 2,3- or 3,5-diiodobenzoic acid (2,3-DIBA, 3,5-DIBA; two iodine atoms at positions R¹ and R² or R⁴ and R⁵ or R³ and R⁵, respectively), 3,5-diiodobenzoic acid (3,5-DIBA; two iodine atoms at positions R₃ and R₅ or R₁ and R₃, respectively), 2,4,6-, 2,3,6-, or 2,3,5-trichlorobenzoic acid (TCBA; three chlorine atoms at the positions R², R⁴, R⁵ or R², R³, R⁵ or R², R³, R⁵, respectively), 3,4,5-, 2,3,4-, 2,3,5-trifluorobenzoic acid (TFBA; each three fluorine atoms at the positions R³, R⁴, R⁵ or R², R³, R⁴ or R², R⁴, R⁵, respectively) etc. If R⁶ is isothiocyanate, the positions R², R⁴ and R⁵ comprise bromine atoms, one refers to 2,4,6-tribomophenyl isothiocanate (TBPI).

According to invention, a conjugate refers to a linkage product consisting of several substances. The substances linked to each other comprise the first and further compounds, if applicable, e.g. second and third compounds as well as the first and further peptides, if applicable, e.g. the second and third peptides. The linkage can be realized by any means, e.g. by a covalent or ionic bond.

The inventors have realized that, due to the X-ray-dense halogens, the conjugate according to the invention results in a particularly well signal when used in imaging methods.

Surprisingly, the inventors have further realized that the first compound complexed with a first peptide can penetrate the membrane of biological cells and can also enter the nucleus. The cell membrane and nuclear membrane penetrativeness of the conjugate according to the invention has the big advantage that the boundaries of a specific tissue or of a tumor, respectively, can be sharply imaged within the context of an imaging method, such as a computer tomography. The conjugate, when administered during or directly after an operation, can no longer run along the space between the cells, opened by the surgeon, beyond the boundaries of the tumor.

In this context, it was particularly surprising that the cell membrane and nuclear membrane penetrativeness of the conjugate according to the invention is achieved without connecting additional large transmembrane transport units, such as e.g. penetratin or transportan. Hereby an unnecessary enlargement of the molecular weight, which could have an influence on the signalling in the computer tomography, is avoided. As a result, the conjugate according to the invention is relatively small, i.e. it ranges within a size of approximately 1,400 Da to approximately 3,300 Da, preferably from 1,600 Da to 1,800 Da.

In contrast to the contrast media described by Torchilin (2000, l.c.) and in the U.S. Pat. No. 5,567,410 the conjugate according to the invention is devoid of a hydrophilic polymeric compound, such as monomethoxypolyethylenglycol (MPEG), polyethylenglycol (PEG), polyvinylpyrrolidon (PVP), etc., which, due to their sizes, would even prevent a penetration of a compound coupled thereto into the interior of biological cells. As a result, the conjugate according to the invention is considerably smaller and has preferably a molecular weight of approximately 0.5 to approximately 5 kDa, further preferably of approximately 1 to approximately 3 kDa, highly preferred of approximately 2 kDa. Instead of a carboxylic acid which serves as a “backbone” in the known contrast media to connect three molecules of TIBA, the conjugate according to the invention comprises a peptide consisting of amino acids linked to each other via peptide bonds. By this measure it is ensured that the conjugate according to the invention can penetrate into biological cells, whereas the known contrast medium remains exclusively in the blood and cannot even reach the interstitial space. The conjugate according to the invention, in contrast to the known contrast medium, further comprises a very high content of halogens, up to approximately 40% in relation to the whole conjugate, is non-toxic for the whole organism and does not form micelles.

In contrast to the compound which is described in the US 2005/0119470 the functioning of the conjugate according to the invention does not require a peptide which is hybridizable with an oligomeric compound, such as a further peptide or a nucleic acid.

The inventors have realized that the conjugate according to the invention is particularly well qualified for labelling a biopsy position since once administered in the biopsied tissue it remains locally fixed. As a result, a later tracking of the biopsy position is possible without any problems.

Surprisingly, the inventors have realized that the conjugate according to the invention, once it is uptaken into the cells, induces in the latter the programmed cell death, the so-called apoptosis, within a short time. As the inventors have realized e.g. the first compound alone, namely in the form of triiodobenzoic acid (TIBA), is not uptaken into the cells. The induction of apoptosis through the conjugate according to the invention uptaken into the cells occurs even with low concentrations, e.g. in the range of <300 μg conjugate/ml. In contrast, traditional contrast media, such as diatrizoate, ioxaglate, iopromide, iotrolan, induce apoptosis only at very high concentrations, e.g. at 250 mg iodine per millilitre); cf. Zhang et al. (2000), Effects of radiographic contrast media on proliferation and apoptosis of human vascular endothelial cells, The British Journal of Radiology 73, pages 1034 to 1041.

In contrast to the composition known from the WO 2006/069677 A2 the therapeutically useful apoptosis inducing property of the conjugate according to the invention already develops without the presence of a further therapeutically active agent and/or a material for the production of an implantable medical device.

As a result, the conjugate according to the invention comprises an important therapeutic potential which can be used for the treatment of tumor diseases. The uptake into the cells occurs independently of the sodium iodine symporter so that now not only thyroid carcinomas can be treated but also other tumors, such as e.g. prostate carcinomas, brain tumors and the mamma carcinoma.

A radioactive labelling of the halogens is not necessary since the apoptosis of the tumor cells is surprisingly already induced by the accumulation of the compound in the nucleus, which is coupled to the peptide. Therefore, a radioactive strain of the body, in particular of the gastric mucosa, the salivary glands or the lactating breast does not take place. Further, for receiving the therapy it is not required to isolate the patients in protected rooms.

The conjugate according to the invention can be administered during an operation for e.g. 20 minutes into the tumor cavity. In the following the resection cavity is rinsed with buffer solution free of conjugates, to remove excessive conjugate which was not uptaken into the cells. The tumor cells which line the resection cavity, are hereby intracellularily or intranuclearily stained and in comparison to the current iodine contrast media of the interstitial space the boundaries of the tumor can be imaged in a sharp manner. For doing so the computer tomography apparatuses which are currently on the market for a use in the operating room and for an operative resection control can be used, e.g. the mobile CT scanner Philips Tomoscan M, Philips Medical Systems, Eindhoven, Netherlands.

In the case of treating a brain tumor with the conjugate according to the invention due to the blood brain barrier which is intact in the healthy brain parenchyma, the uptake into healthy tissue is prevented, whereas in the brain tumor the blood brain barrier is permeable so that the conjugate can infiltrate and induce apoptosis in an targeted manner. At the same time the higher signal density of the tumor cells in the computer tomography can be interpreted as an indication for tumor apoptosis.

Alternatively to the operative administration of the conjugate according to the invention into the resection cavity a reservoir, e.g. an Omaya reservoir, can be positioned in the tumor. A solution containing the conjugates according to the invention can be infused via this access postoperatively, in several cycles, if applicable.

The first peptide can be attached to the first compound in various ways by means of methods known by the skilled person, e.g. under the use of the carboxyl groups of the compound (R⁶) and the formation of a peptidic bond with a free NH₂-group of the peptide.

The objects underlying the invention are herewith fully solved.

It is preferred if the halogen of the first compound is selected from the group consisting of: Iodine, bromine, fluorine, chlorine and astatine.

By this measure the constructive conditions for the conjugate according to the invention are established in an advantageous manner. These halogens are characterized by their high X-ray density and, therefore, result in particularly well signals in imaging methods.

According to the invention it is preferred if the first compound is selected from the group consisting of triiodobenzoic acid (TIBA), 5-iodobenzoic acid (5-IBA, 5-MIBA), 4-iodobenzoic acid (4-IBA, 4-MIBA), 2,3-diiodobenzoic acid (2,3-DIBA), 3,5-diiodobenzoic acid (3,5-DIBA), 2,5-diiodobenzoic acid (2,5-DIBA), trichlorobenzoic acid (TCBA), trifluorobenzoic acid (TFBA), tribromobenzoic acid (TBBA), tribromphenyl isothiocyanate (TBPI).

The inventors have realized that the mentioned substances are particularly qualified as the first compound to realize the conjugate according to the invention. TIBA has a molecular formula of I₃C₆H₂CO₂H and a molecular weight of 499.81 Da. TIBA is registered under the CAS-number 88-82-4. The benzene ring of TIBA is 3 fold iodized, preferably at the positions 2, 3 and 5 (R¹, R² and R⁴) or 2, 4 and 6 (R¹, R³ and R⁵). The iodination however is also possible at other positions of the benzene ring. IBA/MIBA has the molecular formula IC₆H₄CO₂H and a molecular weight of 248.02 Da. IBA/MIBA is registered under the CAS-number 88-67-5. 2,3-DIBA, 3,5-DIBA, 2,5-DIBA comprise the molecular formulas C₇H₄I₂O₂ and the molecular weights of 373.914 Da. 2,3-DIBA, 3,5-DIBA, 2,5-DIBA are registered under the CAS-numbers 19094-48-5 (3,5) or 14192-12-2 (2,5). TCBA has the molecular formula C₇H₃CL₃O₂ and the molecular weight of 225.45862 Da. TCBA is registered under the CAS-number 50-73-7. TFBA comprises the molecular formula C₇H₃F₃O₂ and the molecular weight of 176.09 Da. TFBA is registered under the CAS-number 121602-93-5. TBPI has the molecular formula C₇H₂Br₃NS and a molecular weight of 371.87158. TBPI is registered under the CAS-number 22134-11-8. TBBA has the molecular formula C₇H₃Br₃O₂ and a molecular weight of 358.81 Da. TBBA is registered under the CAS-number 633-12-5.

It is preferred if the first peptide of the conjugate according to the invention comprises a positive net charge.

Net charge refers to a charge of the first peptide which results under physiological conditions (pH 7) from the contribution to the charge of the individual positively or negatively charged amino acid residues of the peptide. The inventors have surprisingly realized that the capability of the conjugate according to invention to penetrate the cell membrane and nuclear membrane can be established in a particularly well manner if the first peptide comprises a positive net charge. In other words, the first peptide preferably comprises such amino acids which are positively charged under physiological conditions. Therefore, the first peptide according to the invention preferably comprises one or several molecules of the “basic” amino acids arginine (R), lysine (K) or histidine (H).

It is preferred if the first peptide of the conjugate according to the invention comprises 2-20, further preferred 5-10 and highly preferred seven amino acids.

Surprisingly, the inventors have realized that such short peptides are sufficient to mediate the transportation of the conjugate both through the cell membrane as well as through the nuclear membrane. As so far assumed in the art large transportation peptides, such as penetratin or transportan, are not necessary. Due to the small size of the first peptide the relation between TIBA which provides a signal in the imaging methods, and the first peptide which does not provide a signal, is kept as low as possible, and a good signal is even given by a small amount of conjugate.

It is further preferred, if the first peptide is derived from a nuclear localization sequence (NLS).

Nuclear localization sequences (NLS) have first been described by Kalderon et al., A short amino acid sequence able to specify nuclear location. Cell 39, 499-509 (1984). They allow larger cyto-plamatic proteins the infiltration into the nucleus through the small nuclear pores. The NLS sequences are recognized by importin-alfa and -beta, resulting in an opening of the nuclear pores for the large proteins of the cytoplasm. For the realization of the conjugate according to the invention the inventors have exemplarily used the NLS of the SV 40 T-antigene and picked out seven consecutive amino acids.

It was of particular surprise that for a mediation of the capability to penetrate the nuclear membrane it was not required to use an exact NLS. The capability of the conjugate according to the invention to penetrate the nuclear membrane was also achieved by the use of a mutated NLS. An identity of the sequence with a natural NLS of 20% or 40%, preferably 60%, more preferred 80%, highly preferred 90%, was already sufficient. It is decisive that at least one or several of the “basic” amino acids contained in the natural NLS remain present.

With the conjugate according to the invention it is preferred if the first compound is bound to the peptide via its carboxyl group.

This measure has the advantage that such a group at the benzene ring of the first compound is used which is particularly qualified, without involving the halogen substitutes into the bond or without sterically interfering or insulating the latter, so that they are available for the realization of the invention.

It is further preferred if the first peptide comprises a free amino function in a side chain via which it is bound to the first compound.

This measure has the advantage that also for the first peptide a particularly well qualified reactive group is used via which the coupling to the first compound is enabled, e.g. through a reaction with the carboxyl group and the formation of a peptide bond. Free amino functions can be found e.g. in the side chain of asparagine (N), glutamine (Q) or lysine (K), and at the N-terminus of the peptide.

It is particularly preferred if the peptide comprises an ε-amino function of a C-terminally located lysine moiety, via which it is bound to the compound.

This measure has the advantage that via the ε-amino function of the terminal lysine moiety the first peptide is bound to the first compound in a particularly efficient manner. The conjugate according to the invention then takes a particularly advantageous conformation which ensures the imaging and apoptosis inducing properties. The terminal lysine moiety can either be part of the first peptide or can also be attached to a terminal end of the first compound as a kind of “hanger”.

According to a preferred embodiment of the invention the first peptide comprises the amino acid sequence PKKKRKV (SEQ ID NO: 1) or PKKTRKV (SEQ ID NO: 2) or PLGLA (SEQ ID NO: 13).

In this presentation the C-terminus is located on the right side and the N-terminus at the left side. The common one-letter code for amino acids is used. This measure has the advantage that the constructive conditions for the first peptide are established, which comprises a positive net charge and which is derived from the NLS sequence of the SV 40 T antigen. The sequence PKKKRKV was identically overtaken from the NLS of the SV 40 T antigen, whereas in the sequence PKKTRKV at the fourth position in relation to the NLS of the SV 40 T antigen the lysine (K) was replaced by a threonine (T). Surprisingly the inventors have realized that also such an NLS sequence which is modified in relation to those of the SV 40 T antigen, mediates the function according to the invention and the capability of the conjugate to penetrate the cell membrane and the nuclear membrane is ensured. It shall be understood that also the other positions in the NLS of the SV 40 T antigen can be replaced without effecting the function of the conjugate according to the invention, as long as the capability to penetrate the cell membrane and the nuclear membrane is maintained. Consequently, first peptides comprising sequences which have a homology of 80%, 85%, 90%, 95%, 98% in relation to SEQ ID NO: 1 or NO: 2, are also qualified. In order to attach the first compound a lysine moiety can be easily provided e.g. at the C-terminus, e.g. resulting in the following sequences PKKKRKVK or PKKTRKVK, whereas the lysine “hanger” is shown by an italic letter.

It is further preferred if the conjugate according to the invention comprises a detectable marker.

This measure has the advantage that due to this further marker the conjugate according to the invention cannot only be tracked by means of the computer tomography but also by means of conventional imaging methods in vivo, in situ but also in vitro, if applicable. As a result, the boundaries of a tumor can also be detected by means of conventional and operative imaging methods, e.g. near infrared imaging. According to the invention, a detectable marker refers to any compound which can be identified by means of imaging methods. This applies to color-indicators having fluorescent, phosphorescent or chemiluminescent properties, dansyl or coumarin dyes, AMPPD, CSPD, non-radioactive indicators, such as biotin or digoxigenin, alkalic phosphatase, peroxydase etc. In exceptional cases also radioactive indicators, such as P³², S³⁵, I¹³², I³¹, C¹⁴ or H³ can be used, however under consideration of the disadvantages in connection with the radioiodine therapy which are mentioned further above. According to the nature of the marker the imaging methods can be microscopic, blotting, hybridization technologies or autoradiography.

It is particularly preferred if the detectable marker is a fluorescent dye, preferably fluorescein isothyocyanate (FITC).

Most of the operation rooms are equipped with fluorescent microscopes. Therewith, after the administration of the conjugates according to the invention into the tumor cell the boundaries of the tumor can already be imaged during the operation. This enables an even better localization of the tumor and maybe better therapeutic or surgical measures.

It is preferred if the detectable marker comprises a free amino function of an amino acid, preferably an ε-amino function of a lysine moiety, via which it is bound to the peptide.

The lysine moiety can either be part of the first peptide or can be attached to a terminal end, preferably C-terminal end, of the first peptide as a kind of “hanger” for the detectable marker. This measure has the advantage that the detectable marker is bound to the conjugate according to the invention in a particularly efficient manner, without negatively effecting its capability to penetrate the cell membrane and nuclear membrane or its property with regards to the contrast conferment and the induction of the apoptosis.

It is preferred if an amino acid spacer is located between the detectable marker and the first compound, which preferably comprises 2 amino acids, further preferred comprises the amino acid sequence GG (SEQ ID NO: 3).

This measure has the advantage that interfering steric interaction between the detectable marker and the first compound are largely avoided and the conjugate according to the invention, despite the coupled further detectable marker, remains functional. It shall be understood that the amino acid spacer can comprise any amino acid sequence or can also have a length of 1 or 3 amino acids, since it has mainly the function to create a distance between the third peptide or the detectable marker, respectively, and the first compound, whereas however, the sequence GG has been proven as particularly qualified. The spacer can also comprise at its C- and/or N-terminus lysine moieties as “hangers” for the detectable marker. The amino acid spacer can also be replaced by a non-peptidic spacer, as long as the before explained function if assured.

It is preferred if the conjugate according to the invention further comprises at least one component which confers a tumor cell specificity or a specificity for virus-infected cells.

Such a component which can be attached to the conjugate or inserted in the latter by means well known by a skilled person, can be realized e.g. in form of an antibody and/or an aptamer, which comprises a specificity or an affinity for such a cell, e.g. bind to a tumor marker or an infection marker, which are specifically expressed at the surface of a tumor or an infected cell. Such a component can further be realized in form of a synthetic ligand which comprises a specificity for such cells, or by viruses or a component thereof, which are coupled to the conjugate according to the invention and confer it a tropism for tumor cells or virus-infected cells, respectively.

It is furthermore preferred if the component comprises a third peptide which (i) comprises a charge which at least neutralizes the positive net charge of the first peptide, and (ii) which is bound to the conjugate via a second peptide, which comprises an amino acid recognition sequence for a tumor cell or virus specific enzyme. Examples for tumor cell or virus-specific enzymes are matrix metalloproteases (MMP), cathepsines, prostate-specific antigen (PSA), herpes-simplex-virus-protease, human immunodeficiency virus protease, cytomegalovirus-protease, interleukin-1β-converting enzyme.

This measure has the advantage that a tumor cell specificity or a specificity for virus-infected cells is conferred to the conjugate according to the invention in a particularly effective manner. It is known in the art that several tumor or carcinoma cells, respectively, express characteristic enzymes and secrete them into their cellular environment, e.g. to digest the surrounding connective tissue to invade the so far healthy tissue or organs. For example glioblastoma express and increase predominantly matrix metalloproteinase 2 (MMP2). Mamma carcinoma express and secrete predominantly cathepsines which recognize and cleave a specific amino acid sequence. Prostate carcinoma may express and secrete prostate-specific antigen (PSA). It is also known that virus-infected cells express and secrete virus-specific proteases. Herpes-simplex-virus (HSV)-infected cells secrete herpes-simplex-virus-protease. Cells which are infected by the HIV virus express and secrete HIV proteases. Cells which are infected by the cytomegalovirus express and secrete a protease which is specific for this kind of virus.

All of the before mentioned enzymes comprise a substrate specificity, i.e. defined amino acid sequences are recognized as cleavage sites. MMP2 recognizes the sequences PLGVR (SEQ ID NO: 4), PLGVA (SEQ ID NO: 5) or PLGLA (SEQ ID NO: 13), whereas cathepsine B recognizes the specific sequences KK (SEQ ID NO: 6) and/or RR (SEQ ID NO: 7). Cathepsine D recognizes the sequence PIC(Et)FF, whereas “Et” refers to an ester branch. Cathepsine K recognizes the specific sequence GGPRGLPG (SEQ ID NO: 8). PSA, on the other hand, recognizes the amino acid sequence HSSKLQ (SEQ ID NO: 9). Further tumor cells specific enzymes and their specific recognition and cleavage sites are well described in the art. An overview on this is given in Hahn, W. C. and Weinberg, R. A., Rules for making human tumor cells, N. Engl. J. Med. 347, pages 1593-1603 (2002), whereby the content of this document is incorporated herein by reference.

The HSV protease recognizes the amino acid sequence AEAGALVNASSAAHVDV (SEQ ID NO: 10), the HIV protease recognizes the sequence SQNYPIVQ (SEQ ID NO: 11), the cytomegalovirus protease recognizes the sequence GVVNASCRLA (SEQ ID NO: 12).

All of the before mentioned sequences can be used as the second peptide or as a component of the second peptide, via which the component or the third peptide, respectively, is bound to the conjugate according to the invention. Second peptides with sequences which comprise a homology of 80%, 85%, 90%, 95%, 98% with the before identified sequences, are also qualified.

The third peptide of the component, which comprises a charge which at least neutralizes the positive net charge of the first peptide, is of particular importance for the tumor cell specificity of the conjugate or the specificity for virus-infected cells, respectively. In order to neutralize the positive net charge of the first peptide the third peptide comprises a negative net charge which compensates the positive charge of the first peptide. The negative net charge of the third peptide can be realized by e.g. negatively charged amino acids, such as glutamic acid (E) or aspartic acid (D).

“At least neutralizing” in this connection means that by the third peptide also a negative net charge of the modified construct according to the invention can result. As a consequence of this neutralization or negativation of the charge of the so modified conjugate according to the invention the latter accumulates in the interstitial space and is no longer in a position to enter the cytoplasm or the nucleus of non-transformed healthy cells. In the neighborhood of the tumor cells, however, the situation is different. Here the above-mentioned tumor cell specific extracellular proteases can be found, which cause the cleavage of the second peptide which comprises the corresponding recognition sequence for these proteases. As a result, in the neighborhood of tumor cells or virus-infected cells, respectively, the third peptide loses its neutralizing or negative charge, respectively. Consequently due to the positively charged first peptide again a positive net charge is prevailing, as a result of this the conjugate according to the invention can penetrate both the cell membrane as well as the nuclear membrane of the tumor cells or virus-infected cells, respectively. This process, therefore, depends on the neighborhood of a tumor cell or virus-infected cells, respectively, so that the modified conjugate according to the invention can only enter into such cells and exert there its apoptopic effect.

Alternatively the component comprises the following, namely a second compound comprising the following formula:

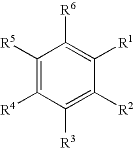

wherein R¹, R², R³, R⁴ and R⁵, independently from each other, each correspond to a halogen or a hydrogen, and R⁶ to a carboxyl group (COOH) or to isothiocyanate (S═C═N), and a second peptide bound to the second compound, which comprises an amino acid recognition sequence for a tumor cell or virus specific enzyme, wherein the component is bound to the conjugate via the second peptide.

Also by this measure the tumor cell specificity or the specificity for the virus-infected cells, respectively, of the conjugate according to the invention, is ensured. The inventors surprisingly herewith provide such a conjugate which blocks itself in its capability to enter the cytoplasm or the nuclei of healthy cells. Such a self-blockade is ensured by the presence of the second compound. Due to the size and configuration of the conjugate resulting therefrom the uptake into the cytoplasm or the nucleus, respectively, of healthy cells, is prevented. The conjugate rather remains in the interstitial space and is excreted from the organism after a while. However, in the neighborhood of tumor cells or virus-infected cells, respectively, the second specificity-mediating peptide is recognized and cleaved by tumor or virus specific proteases, respectively. As a result, the second compound is cleaved off from the conjugate and the latter, due to the first peptide, can enter the cytoplasm and the nucleus of the tumor cell. The cleaved off second component after the cleavage by the tumor or virus specific proteases remain in the interstitial phase for a while and effectively contributes to the signaling in the tumor or the infected area in an advantageous manner, and is then eliminated from the interstitial space and excreted from the organism. It is of particular advantage that the cleaved off second compound does not comprise any neurotoxic properties as this is e.g. described for peptides comprising negative net charge; cf. Garattini et al. (2000), Glutamic Acid, Twenty Years Later, J. Nutr. 130 (4S Suppl.): 901S-9S.

The conjugate according to the invention modified in such a manner is, so to speak, “activated” and becomes cell membrane and nucleus membrane penetrative, whereas such an activation is missing in the presence of healthy cells. This process is highly selective and specific, so that the modified conjugate according to the invention develops its properties exclusively in tumor cells or virus-infected cells, respectively.

According to the invention it is preferred if the halogen of the second compound is selected from the group consisting of iodine, bromine, fluorine, chlorine and astadine.

These halogens are characterized by their high X-ray density and, therefore, provide particularly well signals in imaging methods.

According to the invention it is preferred if the second compound is selected from a group consisting of: triiodobenzoic acid (TIBA), 5-iodobenzoic acid (5-IBA, 5-MIBA), 4-iodobenzoic acid (4-IBA, 4-MIBA), 2,3-diidobenzoic acid (2,3-DIBA), 3,5-diiodobenzoic acid (3,5-DIBA), 2,5-diiodobenzoic acid (2,5-DIBA), trichlorobenzoic acid (TCBA), trifluorobenzoic acid (TFBA), tribromobenzoic acid (TBBA), tribromphenyl isocyanate (TBPI).

As explained above in connection with the first compound the inventors have realized that these substances are particularly qualified for the realization of the second compound.

According to the invention it is preferred if the second peptide comprises the amino acid sequence PLGLR (SEQ ID NO: 4) and/or PLGVA (SEQ ID NO: 5) and/or PLGLA (SEQ ID NO: 13).

This measure has the advantage that such a conjugate is provided by means of which brain tumors can be imaged or treated, respectively, in a specific and highly selective manner. The indicated amino acid sequences are recognized by the matrix metalloprotease 2 (MMP-2) which is characteristic for brain tumors. Second peptides comprising sequences which comprise a homology of 80%, 85%, 90%, 95%, 98% with the SEQ ID NO: 4 or NO: 5 are also qualified. By such a conjugate it is also possible to control the effect of MMP-2-inhibitors, such as Prinomastat, in the antiangiogenesis therapy of glioms by computer tomography.

It is preferred according to the invention if the second peptide is bound to the second compound via the ε-amino function of a C-terminally located lysine moiety.

By this measure the constructive conditions for a stable attachment of the second compound, e.g. TIBA, are established. The lysine moiety can be part of the second peptide, but can also be attached to the C- or N-terminus so that e.g. the following sequences of the second peptide are obtained: PLGLRK or PLGVAK, respectively, whereas the lysine “hanger” is shown in italic letters. Here, the lysine moiety can be preferably covalently bound to the second peptide via its α-amino group or its α-carboxyl group.

According to a preferred further development the conjugate according to the invention comprises a third peptide which preferably comprises a positive net charge, further preferably 2 to 20, preferably 5 to 10, further preferably 7 amino acids. It is further preferred if the third peptide is derived from a nuclear localization sequence (NLS). The third peptide preferably comprises a free amino function of a side chain via which it is bound to the second compound, preferably via an ε-amino function of a lysine moiety which is located at the N-terminus. It is preferred if the third peptide comprises the amino acid sequence PKKKRKV (SEQ ID NO: 1) or the amino acid sequence PKKTRKV (SEQ ID NO: 2).

The third peptide has, therefore, the same characteristics as the first peptide. The explanations given for the first peptide, therefore, apply to the third peptide correspondingly. For example, also to the third peptide a detectable marker, such as FITC, but also another colorant, such as rhodamine, can be coupled in a corresponding manner, so that the marker can be distinguished from each other. This measure creates a more or less symmetric tumor specific conjugate, after cleaving the second peptide by a tumor cell specific protease, such as MMP-2, both cleavage products can specifically enter the tumor cell.

According to an embodiment according to the invention the tumor cell specificity mediating component comprises a nucleic acid molecule which, under stringent conditions, hybridizes to tumor cell specific molecules, preferably to oncogenes.

Such a nucleic acid molecule can be bound to the conjugate according to the invention by several ways by means known to the skilled person. A coupling is possible either to the first and/or second peptide or to TIBA. Here the nucleic acid sequence is selected in such a manner that it is largely complementary to the nucleic acid sequence of tumor cell specific molecules, such as e.g. the coding sequence of an oncogene. By this measure such a nucleic acid molecule can function as a kind of “anchor” and accumulates the conjugate according to the invention in the tumor cells that only there it can develop its activity. To the contrary, no accumulation of the construct according to the invention occurs in non-transformed healthy cells since tumor specific molecules are not expressed there or only to a very small degree. The nucleic acid molecule can be designed in such a manner that it either hybridizes to the mRNA or also to the DNA, which encode the tumor cell specific molecules. An overview on known oncogenes which are involved in the growth of tumors, and from which sequences for the realization of the nucleic acid molecule can be derived, can be found in Vogelstein, B. and Kinzler, K. W., Cancer genes and pathways day control, Nat. Med. 10, pages 789-799 (2004). The content of this document is incorporated herein by reference.

Another subject matter of the present invention relates to the use of the before described conjugate for the production of a diagnostic composition, wherein it preferably relates to a contrast medium for the computer tomography (CT) and/or radioiodine therapy.

Another subject matter relates to the use of the conjugate according to the invention for the production of a therapeutic composition, which is preferably an apoptosis-inducing composition.

Another subject matter relates to a pharmaceutical and/or diagnostic composition which comprises the conjugate according to the invention and, if applicable, a pharmaceutical and/or diagnostic acceptable carrier.

Diagnostic and pharmaceutical acceptable carrier and, if applicable, further additives are generally known in the art and are e.g. described in the publication of Kibbe A., Handbook of Pharmaceutical Excipients, 3^{rd} edition, American Pharmaceutical Association and Pharmaceutical Press 2000. In this category fall e.g. binders, disintegrants, lubricants, salts and further compounds which can be used in the formulation of medicaments.

Another subject matter of the present invention relates to a method for the diagnostic and/or analytical treatment of biological material or a living being, which comprises the following steps: (a) incubation or administration of the conjugate according to the invention with biological material or into a living being, (b) performing an imaging method.

An imaging method generally refers to nuclear medical or radiological methods, including angiography, positron emission tomography, scintigraphy, as well as near infrared imaging or conventional fluorescence microscopy, wherein a computer tomography (CT) is preferred.

Another subject matter of the present invention relates to a method for therapeutically treating a living being, where the conjugate according to the invention is administered into the living being.

It shall be understood that the features mentioned above and those to be explained below cannot only be used in the combination given, but also in other combinations or in isolation without leaving the scope of the present invention.

In the following, embodiments of the invention are explained which are of pure illustrative character and do not limit the scope of the invention. Reference is made to the enclosed figures.

### BRIEF DESCRIPTION OF THE DRAWINGS

FIG. 1 exemplarily shows the ESI mass spectra for the conjugates 1-4 with the molecular masses of 2123.4 Da (K1) (A), 2096.3 Da (K2) (B), 1641.8 Da (K3) (C) and 1614.6 Da (K4) (D).

FIG. 2 shows fluorescence and transmission light microscopic images of human malignant U373 glioma cells after an incubation for 20 minutes with PBS alone (native, column 1, TIBA-containing conjugate 2 (mutant NLS) (126 μM, 260 μM, 2.6 mM, columns 2-4), and non-TIBA-containing conjugate 4 (mutant NLS) (26 μM, 260 μM, 2.6 mM, columns 5-7).

First column (FITC channel): localization of the FITC-labeled conjugate. Untreated cells show no autofluorescence.

Second column: (propidium iodide (PI) channel): Result of the PI viability test. The nuclei of dead cells are stained. The living cells remain dark.

Third column: Result of the MTT viability test. Living cells oxidize methyl-thiazoyl-tetrazolium (MTT) salt, producing blue formazan granules, which form long crystals with increasing duration of incubation.

Fourth column: Superimposed FITC, PI, and formazan images clearly demonstrate that most of the cells that have taken up conjugate 4 in the μmolar and mmolar range are alive (production of formazan, no PI within the cell nuclei). By contrast, all the cells that have taken up conjugate 2 at a concentration of 260 μM or more are non-viable (lack of formazan production, nuclear uptake of PI). An uptake of PI in healthy viable cells with formazan production is possible after a mechanical damage of the cell membrane has taken place due to the long formazan crystals.

FIG. 3A) FACS (fluorescent activated cell sorting) analysis demonstrating a low percentage of strongly labeled cells after incubation with the non-TIBA-containing conjugates (conjugate 3: 26 μM/6%, 260 μM/7% and 2.6 mM/10%) (conjugate 4: 26 μM/4%, 260 μM/11% and 2.6 mM/13%).

FIG. 3B) An obvious increase in heavily stained cells to more than 90% was observed after incubation with the TIBA-containing conjugates (conjugate 1: 26 μM/32%, 260 μM/32% and 2.6 mM/93%) (conjugate 2: 26 μM/0%, 260 μM/78% and 2.6 mM/86%).

FIG. 3C) After the incubation with the TIBA-containing conjugates at 260 μM and 2.6 mM two cell populations could be distinguished on the basis of their morphology (side scatter vs. forward scatter FACS analysis).

FIG. 4A) CLSM images of human malignant U373 glioma cells. The annexin-V-Alexa™ 568 reagent was used to detect phosphatidyl serine in the outer membrane leaflet of necrotic or apoptopic cells. Incubation with either TIBA alone or the non-TIBA-containing conjugates 3 and 4 alone did not result in binding of the annexin-V-Alexa™ 568 reagent to the surface of the glioma cells. The co-incubation of TIBA with conjugates 3 and 4 also fails to result in binding of the annexin-V-Alexa™ 568 reagent to the surface of the glioma cells and was not associated with a higher cellular staining rate. However, binding of annexin-V-Alexa™ 568 reagent was found after incubation with the TIBA-containing conjugates 1 and 2. No signs of cell death were observed after incubation with either PBS or 0.1% DMSO/PBS alone (CLSM images not shown).

FIG. 4B) Overview images (CLSM) of human malignant U373 glioma cells. A very large number of cell nuclei has been stained by the FITC-labeled TIBA-containing conjugates 1 (row 1) and 2 (row 2) (260 μM) (left column). Most of these cells show the expression of phosphatidyl serine in the outer membrane leaflet and are therefore labeled by the annexin-V-Alexa™ 568 reagent (right column).

FIG. 4C) Fluorescence microscopy of semi-thin sections (about 0.4 μm) of human malignant U373 glioma cells after the incubation with the TIBA conjugates 1 (correct NLS) and 2 (mutant NLS). The nucleoli of the cells are clearly stained.

FIG. 5A) Representative CT image of human malignant U373 glioma cells with only low signal density values after the incubation (20 minutes) with PBS alone (tube 1), Ultravist (iopromide) (260 μM) (tube 2) TIBA alone (260 μM) (tube 3) and conjugate 2 (tube 4) and 1 (tube 5) alone (both 26 μM). A significant increase in signal density was only observed after the incubation with the TIBA-containing conjugate 2 and 1 at higher concentrations [conjugate 2: 260 μM (tube 6) and conjugate 1: 260 μM (tube 7)] [conjugate 2: 2.6 mM (tube 8) and conjugate 1: 2.6 mM (tube 9)]. About 6×10⁶ cells per tube were examined. Investigations were performed in triplicate [windowing: −1/74].

FIG. 5B) Corresponding MTT-test results of cell pellets from the computer tomography after the incubation with conjugate 2 (above) and conjugate 1 (bottom) both: 26 μM, 260 μM and 2.6 mM). Only living cells oxidize the yellow methyl-thiazoyl-tetrazolium (MTT) salt to blue formazan (incubation with either PBS alone or both conjugates at 26 μM).

FIG. 5C) CT image (InSpace) of human malignant U373 glioma cells after the incubation (20 minutes) with PBS alone (left), TIBA-containing conjugate 1 (correct NLS) (middle) and TIBA-containing conjugate 2 (mutant NLS) (right) (both 2.6 mM). No substantial differences between conjugates 1 and 2 were seen.

FIG. 5D) Corresponding signal density values for cell pellets in tubes 1-9 (partial Fig. A).

FIG. 6A) Above: CT image of human prostate cancer cells (PC3) after the incubation (20 minutes) with the non-TIBA-containing conjugate 4 (mutant NLS) (260 μM, left) and the TIBA-containing conjugate 1 (correct NLS) (260 μM, right) (windowing: −2/112). Bottom: Fluorescence and transmission light microscopy images of human prostate cancer cells (PC3) after the incubation for 20 minutes with PBS alone (native, row 1), non-TIBA-containing conjugate 3 (correct NLS) (520 μM, row 2) and the TIBA-containing conjugate 2 (mutant NLS) (520 μM, row 3).

FIG. 6B) Prostrate cancer cells (PC3) FACS (fluorescence activated cell sorting) analysis which is similar to that of the human malignant U373 cells.

FIG. 6C) After the incubation with the TIBA-containing conjugates at 260 μM and 2.6 mM two cell populations could be differentiated on the basis of their morphology (side scatter vs. forward scatter FACS analysis). The upper cloud (image right, top) represents the population of strongly stained cells (high histogram peak on the right; image left, top). The cloud at the bottom (image to the right, bottom) represents the population of weakly stained cells (flat histogram peak at the left; image left, bottom).

FIG. 7 Confocal laser microscopy of human malignant LN18 glioma cells. The annexin-V-Alexa™ 568 reagent was used to detect phosphatidyl serine in the outer membrane leaflet of necrotic or apoptotic cells. The co-incubation of either MIBA (4-monoiodinebenzoic acid) or DIBA (2,5-diodinebenzoic acid) with conjugate 3 did not result in a binding of annexin-V-Alexa™ 568 to the surface of the glioma cells and, in comparison with the incubation with conjugate 3 alone, did not result in a higher staining rate.

FIG. 8 Confocal laser microscopy of human malignant LN18 glioma cells. The incubation with the conjugates 3 and 9 (260 μM) did only result in a nuclear staining of few cells. These remain vital (no annexin-V-Alexa™ 568 reagent staining). However, a high percentage of cells with signs of cell death (annexin-V-Alexa™ 568 reagent staining) could be found after the incubation with the MIBA (4-monoiodinebenzoic acid) and DIBA (2,5-diiodinebenzoic acid) containing conjugates 10 and 11 (260 μM).

FIG. 9 FACS (fluorescence activated cell sorting) analysis. A clear increase of the strongly stained cells (over 80%) could only be found after the incubation with the MIBA- (4-monoiodinebenzoic acid) and DIBA- (2,5-diiodinebenzoic acid) containing conjugates 10 and 11 (260 μM). This can be seen from the shift of the histogram peak to the right.

FIG. 10 CLSM (confocal laser microscopy). U373 glioma cells after the incubation with conjugate 12: Many nuclearly stained nuclei; left top: FITC channel: determination of the localization of the conjugate; right top: annexin channel for the staining with annexin-Alexa as an indication for a strong expression of phosphatidyl serine at the cell death; left bottom: transmission microscopy; right bottom: superposition of the FITC and annexin channels.

FIG. 11 FACS (fluorescence activated cell sorting) analysis after the incubation of the LN18 and U373 glioma cells with the conjugate 12 (260 μM and 2.6 mM). At the higher concentration clearly more cells are stained in both cell lines. This staining is stronger in the U373 glioma cells. In the cloud diagram at the higher concentration two morphologically different populations can be observed.

FIG. 12 CT image (InSpace mode) of the LN18 and U373 glioma cells after the incubation with conjugate 12 (260 μM and 2.6 mM). Only after the incubation at the higher concentration the cell centrifugates can be delimited in the tubes due to the higher signal density.

FIG. 13 Confocal laser microscopy of the LN18 glioma cells. The incubation of free non-coupled trifluorobenzoic acid (TFBA) in combination with conjugate 3 does only result in few stained cells which remain vital [no cellular uptake of propidium iodine (PI)]. However, the incubation with conjugate 13 (trifluorobenzoic acid is tightly coupled to K3) stains a large number of cells in the nucleus (left column). These cells do now uptake PI and are no longer vital (second column).

FIG. 14 CLSM of LN18 glioma cells. After the incubation with conjugate 3 in combination with free trichlorobenzoic acid only few cells stained in their nuclei can be found. These remain vital (no staining with annexin-Alexa, column 2). Only after the incubation with the conjugate 14 (TCBA is tightly coupled to K3) almost all nuclei are stained. The cells are dead (staining with Alexa-annexin).

FIG. 15 Confocal laser microscopy of LN18 glioma cells. The incubation with free tribromophenylisocyanate (TBPI) only does not result in a cell death (no annexin-Alexa staining, 2. column). The TBPI-free conjugate 3 does only stain the nucleus of few cells which remain vital (no staining with annexin). Also the co-incubation of free TBPI and conjugate 3 results only in the staining of the nucleus in few cells without an impairment of the vitality (no staining with annexin). Only after the incubation of K15 (TBPI tightly bound to K3) a strong staining of the nucleus in almost all cells can be shown (column 1). Alexa-annexin binds to the surface of the cells (2. column, indication of cell death).

FIG. 16 A: confocal laser microscopy (superposition of the FITC and Alexa images). The nuclei of the LN18 glioma cells accumulate the FITC-labeled tribromophenyl isocyanate-NLS-conjugate (K15). The red Alexa-annexin binds to phosphatidyl serine which is strongly expressed at the cell surface during the cell death. B: FACS (fluorescence activated cell sorting) analysis of LN18 and U373 glioma cells after the incubation either with conjugate 3 alone, conjugate 3 in combination with free tribromophenylisocyanate (TBPI) or the TBPI-NLS-conjugate 15. A clear increase of the cells which are strongly stained by FITC (shift of the histogram peak to the right) can only be shown after the incubation with the conjugate 15. C: Semi-thin section of a LN18 glioma cell. The nucleolus (in the center) is strongly stained by the tribromophenylisocyanate-NLS conjugate 15.

FIG. 17 FACS (fluorescence activated cell sorting) analysis of LN18 and U373 glioma cells. The incubation of the conjugates 13 [trifluorobenzoic acid (TFBA) coupled to K3] or 14 [trichlorobenzoic acid (TCBA) coupled to K3] in each case results in a shift of the histogram peak to the right and therefore in an increase of the number of cells which are strongly stained by FITC. However, if free unbound TFBA or TCBA, respectively, is incubated in combination with the conjugate 3 no shift of the histogram peak to the right can be observed.

FIG. 18 Mode of action of the tumor specific conjugate 16.

FIG. 19 Confocal laser microscopy of LN18 glioma cells after the incubation with conjugate 16 in the presence of inactive (upper row) or active matrixmetalloproteinase 2 (MMP-2). The nuclei are only stained by the cleaved conjugate after the action of activated MMP-2 (left column). Only the cleaved conjugate can induce the cell death (image of propidium iodine, second column).

FIG. 20 FACS (fluorescence activated cell sorting) analysis of LN18 glioma cells after the incubation with the conjugate 16 in culture medium in each case with inactive or active matrixmetalloproteinase 2 (MMP-2) at 65 and 260 μM. After the cleavage of the conjugate 16 by the active MMP-2 the LN18 glioma cells are stained clearly stronger (shift of the histogram peak to the right).

FIG. 21 CT image (InSpace mode). Left tube: LN18 glioma cells (native, incubation in medium without conjugate 16 for 60 minutes). Middle tube: LN18 glioma cells after incubation with the conjugate 16 (260 μM) in MMP-2 containing medium for 60 minutes. The MMP-2 was inhibited by the MMP-2 inhibitor I. Right tube: LN18 glioma cells after an incubation with K16 (260 μM) in MMP-2 containing medium for 60 minutes. The active MMP-2 was not inhibited by the MMP-2 inhibitor I.

FIG. 22 HPLC (High Performance Liquid Chromatography) of conjugate 16 before (A) and after (B) the influence of the matrixmetalloprotease 2 (MMP-2). Before the cleavage of the conjugate 16 only one peak can be found which separates after the cleavage into two components.

### DESCRIPTION OF PREFERRED EMBODIMENTS

### 1. Material and Methods

### 1.1 Peptide Synthesis

### 1.1.1 Conjugates 1 to 8:

The conjugates 1 to 8 (Table 1) were synthesized on an Eppendorf ECOSYN P-solid phase synthesizer employing Fmoc Rink Amid Tentagel SRAM (0.25 mM/g) (Rapp Polymere, Tübingen, Germany). All amino acids (0.1 mM per 0.4 g resin) except the N-terminal proline were incorporated with amino functions protected by the 9-fluorenylmethyloxycarbonyl(Fmoc) group: The side chain functions were protected as tert-butylether (threonine), 2.2.4.6.7.-pentamethyl-dihydrobenzofuran-5-sulfonyl(arginine), tert-butyloxycarbonyl (lysine, except lysine 8) or 4-methyltrityl (lysine-8). Fmoc Lys (N′-2,3,5-triiodobenzoyl) was prepared by coupling of N^{ε}-Fmoc-Lys-OH with 2,3,5-triiodobenzoic acid (TIBA) by activation with isobutylchloroformiat (1 eq.) and N-methylmorpholin (1 eq.) (mixed anhydride coupling). The substance was re-crystallized from DMF/diethylether. All couplings were performed using a fourfold excess of amino acids and the coupling reagents 2-(1-H-benzotriazol-1-yl)-1.1.3.3-tetramethyluronium tetrafluoroborate (TBTU)+diisopropylethylamine (2 eq.) over the amount of resin.

Before the coupling of the protected amino acids, the Fmoc groups were removed from the amino end of the growing segment using 25% piperidine in DMF. The FITC moieties were introduced in the lysine-8 residue with fluorescein-5(6)-isothiocanate in DMSO+N-methylmorpholine (1 eq.) after removal of the 4-methyltrityl group from lysine-8 with TFA in dichloromethan (1%)+triisopropylsilane (1%) for 1 hour at room temperature. The N-terminal proline was incorporated as its Boc derivative. Simultaneous cleavage of the amino acid side chain protecting groups was performed by incubating the resin in a mixture of 12 ml trifluoracetic acid, 0.3 ml ethandithiol, 0.3 ml anisole, 0.3 ml water and 0.3 ml triisopropylsilane for 2 hours. The mixture was filtered and washed with TFA and the combined filtrates were precipitated with anhydrous diethylether.

The crude products were further purified by HPLC on a nucleosile 100 C18 (7 μm) 250×10 column elution being monitored at 214 nm (buffer A: 0.07% TFA/H₂O, buffer B: 80% CH₃CN/0.058% TFA/H₂O; 4 ml/min). The peptides were assayed for purity by analytical high performance liquid chromatography (HPLC) and electrospray ionization mass spectrometry (ESI/MS). Substance purity was at least 98%.

### 1.1.2 Conjugates 9, 10 and 11

Fmoc Lys (N-Benzoyl), Fmoc Lys (N-4-monoiodobenzoyl) and Fmoc Lys (N-2,5-diiodobenzoyl) were produced by coupling N-Fmoc Lys-OH with benzoic acid (BA), 4-monoiodobenzoic acid (MIBA) and 2,5-diiodobenzoic acid (DIBA) (Sigma-Aldrich, Taufkirchen, Germany) [activation with isobutylchloroformiate (iBuOCOCl) (1 eq.) (Merck) and N-methylmorpholine (NMM) (1 eq.) (Fluka, Buchs, Switzerland) (mixed anhydride coupling)]. Besides, the synthesis of the conjugates 9, 10 and 11 was performed in the same manner as described under 1.1.1. The purity of the conjugates (at least 98%) was assayed by means of the analytical HPLC (high performance liquid chromatography). The mass was determined by electrospray ionization mass spectrometry (ESI/MS) (see 1.2).

### 1.1.3 Conjugate 12:

Fmoc Pro (N-triiodobenzoyl) was produced by coupling N-fmoc-Pro-OH with triiodobenzoic acid. The remaining synthesis of the conjugate 12 was performed on an Eppendorf ECOSYN P solid phase synthesizer (Eppendorf-Biotronik, Hamburg, Germany) as described under 1.1.1 [purity of the conjugate at least 98%, analytical HPLC (high performance liquid chromatography)]. The mass was determined by means of electrospray ionization mass spectrometry (ESI/MS) as in section 1 (see 1.2).

### 1.1.4 Conjugates 13, 14, 15

Fmoc Lys (N-benzoyl), Fmoc Lys (N-trichlorobenzoyl), Fmoc Lys (N-2,5-trifluorobenzoyl) and Fmoc Lys (N-2,4,6-tribromophenyl-ureido)-OH were produced by coupling N-Fmoc Lys-OH either with benzoic acid (BA), trichlorobenzoic acid (TCBA), trifluorobenzoic acid (TFBA) (Sigma-Aldrich, Taufkirchen, Germany) or 2,4,5-tribromophenyl isocyanat (TBPI) (Sigma-Aldrich) [activation with isobutylchloroformiate (iBuOCOCl) (1 eq.) (Merck) and N-methyl-morpholium (NMM) (1 eq.) (Fluka, Buchs, Switzerland) (mixed anhydride coupling). The remaining synthesis of the conjugate was performed in the same manner as described under 1.1.1 [purity in the analytical HPLC (high performance liquid chromatography) at least 98%]. The mass was determined by means of electrospray ionization mass spectrometry (ESI/MS) (see 1.2).

### 1.1.5 Conjugate 16

The synthesis of tumor specific conjugate was performed according to the Fmoc solid phase synthesis on an Eppendorf ECOSYN P peptide synthesizer (Eppendorf-Biotronik, Hamburg, Germany). The basic-cleavable 9-fluoroenylmethyloxycarbonyl group was used as amino protection group. Tentagel S rink amid resin (Rapp-Polymere, Tübingen, Germany) was used as carrier material. The synthesis was performed in a 0.1 nMole scale. The couplings were performed with the correspondingly protected Fmoc amino acids at a 4-fold excess with 2 (1H benzotriazol-1-yl)-1.1.3.3-tetramethyluronoium tetrafluoroborat [TBTU] (4 eq.) in the presence of 8 eq. diisopropylethylamine within 40 minutes. As protective groups for the side chains the following were used: For lysine: tert. butyloxycarbonyl (Boc), for arginine: pbf (N-6-2.2.4.6.7-pentamethyldihydro-benzofuran-5-sulfonyl).

For the side chains which are to be provided with triiodobenzoyl groups lysine derivatives with 4-methoxytrityl (Mmt)-protection were used. For the position which should carry the fluorescein urea moiety the Lys-Dde-derivative [Dde=1-(4.4-dimethyl-2,6-dioxocyclohex-1-ylidene)-ethyl] was used.

After the coupling the Fmoc moiety was in each case cleaved by 25% piperidin/dimethylformamid (DMF) solution within 11 min. After several wash steps with dimethylformamide (DMF) the peptide resin can be used for a further coupling.

After the successive assembly of the peptide starting from the C-terminus the N-terminal amino acid proline is introduced into the peptide as Boc-proline. Then the Mmt-side chain protective group is cleaved off within an hour by several additions of 1% TFA/dichloromethane (DCM)-solution which contains 1% triisopropylsilane. After several wash steps with DMF and neutralization of the resulting TFA salt with diisopropylethylamine the exposed side chain is available for a coupling with 2.3.5 triiodobenzoic acid (3 eq. in the presence of 3 eq. TBTU and 6 eq. diisopropylethylamine within 1.5 hours at room temperature). Then the Dde-protective group is cleaved off by several additions of a 2.5% hydrazine hydrate solution in DMF to the resin within an hour.

After several wash steps with DMF the fluorescein urea derivative is produced by coupling 0.5 mM fluorescein 5(6)-isothiocyanate in the presence of the eq. amount of diisopropylethylamine in DMSO over night at room temperature.

After several wash steps with DMF, methanol and dichloromethane after drying the remaining protective group and the peptide are simultaneously cleaved off from the resin. This happens by stirring of the dried resin for three hours in a mixture of 12 ml TFA, 0.3 ml ethandithiol (EDT), 0.3 ml anisole, 0.3 ml water and 0.1 ml triisopropylsilane at room temperature.

Then it is directly filtrated in cooled absolute diethylether. The precipitated peptide is filtrated, washed with ether and dried in the vacuum. The obtained crude peptides are purified by a semi-preparative HPLC under the use of a nucleosil 100 7 mm C18 column (10×250 mm) (buffer A: 0.07% TFA/H₂O buffer B: 80% CH3CN in 0.058% TFA/H2O) (4 ml/min 90 bar, 214 nm); 10® 90% B in 13 min. The obtained conjugate is homogeneous in the analytic HPLC (purity at least 98%) and in conformity with its structure (ESI-MS).

### 1.2 Electrospray Ionization Mass Spectrometry (ESI-MS)

The conjugates were analyzed by ESI-MS on an Esquire3000+ ion trap mass spectrometer (Bruker-Daltonics, Bremen, Germany). The peptides were dissolved in 40% ACN, 0.1% formic acid in water (v/v/v) (20 pmol/μl) and constantly infused using a syringe pump (5 μl/min flow rate). Mass spectra were acquired in the positive ion mode. Dry gas (6 l/min) temperature was set to 325° C., the nebulizer to 20.0 psi, and the electrospray voltage to −3700V.

### 1.3 Cleavage Test (Conjugate 16)

The conjugate 16 was dissolved in HEPES buffer which in one case contained the active MMP-2 (Calbiochem, Bad Soden, Germany) and in the other case the inactive MMP-2 proform (Clabiochem). The incubation in HEPES with active MMP-2 occurred for 2 hours.

For the transformation of the inactive MMP-2 proform into the active MMP-2 APMA (4-aminophenyl mercuric acetate) was used. For this APMA stock solution (100 mM in DMSO) was added to the solution with the proenzyme and the conjugate (final APMA concentration: 1 mM, with 1% DMSO). In the following the conjugate was incubated for 2 hours.

As a control the conjugate 16 was only incubated in HEPES buffer. The tests were also performed with a MMP-2-inhibitor. The cleavage products were evaluated with the HPLC:

Column: Nucleosil 100 5 μm C₁₈ (250×4); buffer A: 0.07% CF₃COOH/H₂O;

### Buffer B: 0.058% CF₃COOH/80% CH₃CN

10→90% B in 36 min; 170 bar; 1 ml/min; 214 nm

### 1.4 Fluorescence Microscopy and Flow Cytometry (Conjugates 1 to 4)

Human malignant U373-glioma cells were grown to 70% confluency in RPMI-1640 Ready Mix Medium containing L-Glutamine and 10% FBS-Gold (PAA laboratories, Pasching, Austria) at 37° C., 5% CO₂ (vol/vol), in 4-well-plates (NUNC, Wiesbaden, Germany) with about 300,000 cells pro well. The cells were incubated with Dulbecco's PBS (D-PBS; GIBCO, Invitrogen, Germany) alone (negative control) and with 26 μmol, 260 μmol and 2.6 mmol solutions of conjugates 1-4 in D-PBS for 20 minutes at 37° C. in an atmosphere of 5% CO₂. After this, the cells were washed three times with buffer and then incubated with Ready Mix Medium again. Cell viability was then assessed by the addition of methyl-thiazoyl-tetrazolium (MTT) salt (Sigma-Aldrich, Germany) at a concentration of 15 mg/ml. After 20 minutes, the production of formazan was investigated. When blue formazan granules were detected, propidium iodine (PI) was added to the medium (1 μM PI; Molecular Probes, Eugene, Oreg., USA) to detect cells with damaged cell membranes. The formazan production was observed for at least two hours for fluorescence and transmission light microscopy an inverted microscope (Axiovert 135 M, Carl Zeiss, Jena, Germany) long-distance (LD) objectives (Carl Zeiss, Jena, Germany), an illuminator N HBO103 (Carl Zeiss, Jena, Germany) and standard fluorescence filters for excitation and emission of FITC and PI were used. Pictures were taken with a 3-CCD color video camera (MC3254P, Sony, Japan) and the Axiovision Software (Carl Zeiss, Jena, Germany). The intensity of cell fluorescence was recorded at the exposure time necessary for the production of the fluorescence images.

After imaging, Accutase™ (PAA laboratories, Pasching, Austria) was added to the wells to achieve detachment of the cells for further FACS analysis. Fluorescence was measured in a Becton Dickinson FACSCalibur. A total of 20,000 events per sample were analyzed. The investigations were performed in triplicate.

### 1.5 Computer Tomography and Flow Cytometry

### 1.5.1 Conjugates 1 to 8

For CT and FACS human U373 glioma cells were grown under the same conditions in 75 cm² culture flasks (Corning Costar, Bodenheim, Germany) (70% confluency). Accutase™ (PAA laboratories, Pasching, Austria) was added to achieve detachment of the cells which were harvested and subsequently aliquoted into Eppendorf tubes (6×10⁶ cells per tube). As in the investigations performed by fluorescence microscopy, the cells in the first tube served as a control (PBS only). The cells in the other tubes were incubated with 260 μM Ultravist, 260 μM TIBA in PBS and 26 μM, 260 μM and 2.6 mM conjugates 1-4 in PBS. After a 20 minute incubation period at 37° C. in an atmosphere of 5% CO₂, the cells were washed three times in PBS and centrifuged at 800 rpm for 5 minutes.

A sample of cells from each Eppendorf tube was subjected to the MTT test (described under 1.4) to determine microscopically whether the cells were viable. In vitro CT of the cell pellets was performed with a Somatom Sensation 16 (Siemens) which is used for routine clinical investigations.

The inner ear spiral CT protocol consisted of: tube voltage 120 KV, effective mAs 550, time of imaging TI: 1.5, SL 0.75/0.75/4.5, FOV: 50 0/52, kernel: U70, window: intervertebral disc.

3D-images were obtained using InSpace Software (Syngo CT 2006G) (Siemens AG, Erlangen, Germany).

The FACS analysis was performed as described under 1.4.

### 1.5.2 Conjugates 9, 10 and 11:

The human malignant U373 and LN18 glioma cells were cultivated in 75 cm² culture flasks (Corning Costar) (70% confluency) under the conditions as described under 1.4, detached from the flask bottom with Accutase™ (PAA Laboratories) and in the following distributed on Eppendorf tubes (Eppendorf, Hamburg, Germany) (6×10⁶ per tube). The cells in the first tube serve as a control (only PBS buffer only). The cells in the other ten tubes were each incubated with benzoic acid (BA), monoiodobenzoic acid (MIBA) or diiodobenzoic acid (DIBA) alone (260 μM), conjugate 3 alone (260 μM), conjugate 3 plus either BA, MIBA or DIBA (260 μM), respectively, and the conjugates 9, 10, and 11 alone. (260 μM).

After an incubation of 20 minutes at 37° C./5% CO₂ the cells were washed three times with PBS buffer and centrifuged at 800 rpm (rounds per minute) for 5 minutes. The fluorescence was measured in Becton Dickinson FACSCalibur as described under 1.4. The computer tomography was performed as described under 1.5.1. The examinations were repeated two times.

### 1.5.3 Conjugate 12:

For the CT and FACS examinations human LN-18 and U373 glioma cells were cultivated in 75 cm² culture flasks (Corning Costar) (70% confluency) (conditions as described under 1.4). Accutase™ (PAA Laboratories) was added to detach the cells from the bottom of the culture flasks. The cells were collected and then distributed on Eppendorf tubes (6×10⁶ cells per tube). The cells in the first two tubes serve as a control (in each case only PBS, LN18 and U373 glioma cells, native). The cells in the other tubes (in each case LN18 and U373 glioma cells) were incubated with a 260 μM or 2.6 mM solution of the conjugate 12 for 20 minutes at 37° C. and 5% CO₂ and in the following washed for three times with PBS buffer and centrifuged for 5 minutes with 800 rpm (rounds per minute). For a small amount of the cells it was tested by the MMT test (see 1.4) how many of the cells were still viable. The computer tomography of the cell centrifuges was performed with the Somatom Sensation 16 (Siemens).

An Orbita CT spiral was used: tube voltage 120 KV, effective mAs 550, time of imaging TI: 1.5, SL 0.75/0.75/4.5, FOV: 50 0/52, GT: 0.0, kernel: U70, window: intervertebral disc. 3D images were made with the InSpace Software (Syngo CT 2006G) (Siemens AG, Erlangen, Germany).

The FACS analysis was performed as described under 1.4. The assay was repeated two times.

### 1.5.4 Conjugate 13, 14 and 15

For the CT and FACS examinations human LN18 and U373 glioma cells were cultivated in 75 cm² culture flasks (70% confluency) (conditions as described under section 1). Accutase™ (PAA Laboratories) was added to detach the cell from the culture bottom. The cells were collected and then distributed on Eppendorf-tubes (6×10⁶ cells per tube). The first 4 tubes were incubated with each of the conjugates 3, 13, 14 and 15 solved in PBS-buffer at a concentration of 260 μM. The next three tubes were used for the coincubation of the cells either with trichlorobenzoic acid (TCBA), trifluorobenzoic acid (TFBA) or tribromophenyl Isocyanat (TBPI) and the NLS-FITC-conjugate 3 (260 μM). As control cells were used which were only either incubated with CIBA, FIBA, TBPI (in each case 260 μM in PBS) or with PBS-buffer (native control) alone. After the incubations the cells were washed three times with PBS-buffer and centrifuged at 800 rpm (rounds per minute). The computer tomography and FACS analysis of the cell centrifuges were performed as described in 1.5.1 or 1.4, respectively for three times.

### 1.5.5 Conjugate 16:

Human U373- and LN18-glioma cells were cultivated in 25 cm² culture flasks (Corning Costar, Bodenheim Germany) (70% confluency) which contained 3 ml RPMI-1640 Ready Mix Medium with L-glutamin and 10% FBS (fetal bovine serum)-gold (PAA laboratories, Pasching, Austria) [37° C., 5% CO₂ (vol/vol)]. Accutase™ (PAA laboratories, Pasching, Austria) was added to detach the cells from the flask bottom. The cells were collected and then distributed on 8 Eppendorf-tubes (6×10⁶ cells per tube). For the transformation of the inactive MMP-2 proform into the active MMP-2 APMA (4-aminophenyl mercuric acetate) was used. For this a 1% APMA-stock solution (100 mM in DMSO) was added to the solution with the proenzyme and the conjugate 16 (final APMA-concentration: 1 mM, with 1% DMSO). The cells in the first four tubes serve as a control (only RPMI-medium with and also without APMA).

The cells in the other four tubes were incubated with 65 and 130 μM of the conjugate 10 for 1 or 2 hours, respectively, either or also without MMP-2 inhibitor I (37° C. and 5% CO₂). The MMP-2 inhibitor I was used as previously described by Yin et al. 2006. After an incubation for 1 or 2 hours respectively, it was three times washed with PBS-buffer and centrifuged at 800 rpm for 5 min. The cell viability was then checked by the aid of methyl-thiazoyl-tetrazolium (MTT) salt (Sigma Aldrich, Germany) (15 mg/ml). The formation of formazan was analyzed after 20 minutes in the transmission microscope.

After blue formazan granula could be detected propidium iodide (PI) was added to the medium (1 μM PI; molecular probes, Eugene, Oreg., USA) to localize cells with damaged membranes. The production of formazan was examined over a time period of at least two hours. For the fluorescence and transmission light microscopy an inverse microscope (Axiovert 135 M, Carl Zeiss, Jena, Germany), a Long Distance (LD) Objective (Carl Zeiss, Jena, Germany) an illuminator N HBO103 (Carl Zeiss, Jena, Germany) as well as a standard fluorescence filter for the excitation and the emission of FITC and PI were used.

The computer tomography of the cell centrifugates was performed with an Somatom Sensation 16 (Siemens) which is also used for clinical routine examinations.

A Felsenbein CT Spiral was used: tube voltage 120 KV, effective mAs 550, time of imaging TI: 1.5, SL 0.75/0.75/4.5, FOV: 50 0/52, GT: 0.0, kernel: U70, window: intervertebral disc. The signal density of each cell pellet was measured.

3D-images were made by the InSpace Software (Syngo CT 2006G) (Siemens AG, Erlangen, Germany). The FACS analysis was performed as described in the following. The assay was repeated two times.

The FACS analysis was performed on a Becton Dickinson FACSCalibur [100 μl of the cell suspension (1×10⁶ cells) plus 300 μl FACS-buffer (D-PBS-buffer with 1% paraformaledhyd)]. About 25,000-35,000 cells were measured per sample [fluorescence excitation: argon ion laser (488 nm), fluorescence detection: 540-565 nm band-pass filter]. The assays were in each case performed in triplicate.

### 1.6 Confocal Laser Scanning Microscopy and Annexin-V-Binding Assay/Viability Test

### 1.6.1 Conjugates 1 to 8

Human malignant glioma cells (U373) were grown in 4-well-plates under the same conditions as for the fluorescence microscopy described under 1.5.

Cells were incubated for 20 minutes with each of the conjugates dissolved in 0.1% DMSO/PBS at 260 μm. The cells were also incubated with TIBA and both of the non-TIBA-containing conjugates (3 and 4) separately in 0.1% DMSO/OBS at 260 μm. Both TIBA and the conjugates were dissolved in 0.1% DMSO/PBS at 260 μm for incubation due to the insolubility of TIBA (but not the conjugates) in pure PBS. As controls, the cells were incubated with PBS alone, 0.1% DMSO/PBS alone, and TIBA alone (260 μm in 0.1% DMSO/PBS).

The detection of phosphatidyl serine in the outer membrane leaflet of apoptotic cells was performed with the Annexin-V-Alexa™-568-reagent according to manufacturer's protocol (Roche Molecular Biochemicals, Indianapolis, USA). The confocal laser-scanning microscopy was performed on an inverted LSM 510 laser-scanning microscope (Carl Zeiss, Jena, Germany) (objectives: LD Achroplan 40×0.6, Plan Neofluar 20×0.50, 40×0.75). For fluorescence excitation, the 488 nm line of an argon-ion laser and the 534 nm line of a helium-neon laser with appropriate beam splitters and barrier filters were used for FITC and Alexa respectively. Superimposed images of FITC- and Alexa-stained samples were created by overlaying coincident views. All measurements were performed on living, non-fixed cells.

### 1.6.2 Conjugates 9, 10 and 11

Human malignant LN18 and U373 glioma cells were cultivated in four-well plates (NUNC, Wiesbaden, Germany) (about 300,000 cells per well) as described under 1.5. The cells were incubated at 37° C./5% CO₂ for 20 minutes with each of the conjugates 3, 9, 10 and 11 at a concentration of 260 μM (dissolved in PBS) (GIBCO; Invitrogen, Germany). The cells were also coincubated either with CIBA, FIBA or TBPI as well as the NLS-FITC conjugate 3 (each 260 μM). As a control cells were used which were only incubated either with CIBA, FIBA or TBPI (260 μM in PBS) alone. After the incubation the cells were washed three times with PBS and in the following again incubated in Ready Mix Medium. The FITC-labelled conjugates and the Alexa-Annexin for the detection of phosphatidylserine as a signal of the cell death were localized with the confocal laser microscopy (CLSM) as in 1.6.1. All measurements were performed on living cells.

### 1.6.3 Conjugate 12:

Human malignant LN18 and U373 glioma cells were cultivated in four-well plates (NUNC, Wiesbaden, Germany) (about 300,000 cells per well), as described in 1.5. The cells were incubated at 37° C./5% CO₂ for 20 minutes with the conjugate 12 at concentrations of 260 μM and 2.6 mM (dissolved in PBS-puffer) (GIBCO; Invitrogen, Germany). As a control cells were only incubated with PBS-buffer. After the incubations the cells were washed for three times with PBS and in the following again incubated in Ready Mix Medium. The detection of phosphatidyl serine and CLSM occurred as described in 1.6.1. All measurements were performed on living cells in triplicates.

### 1.6.4 Conjugates 13 to 15

Human malignant LN18 and U373 glioma cells were cultivated in four-well plates (NUNC, Wiesbaden, Germany) (about 300,000 cells per well) as described in section 1. The cells were incubated at 37° C./5% CO₂ for 20 minutes with each of the conjugates 3, 13, 14 and 15 at a concentration of 260 μM (dissolved in PBS) (GIBCO; Invitrogen, Germany). The cells were also coincubated either with CIBA, FIBA or TBPI as well as the NLS-FITC conjugate 3 (each 260 μM). As a control cells were used which were merely either incubated with CIBA, FIBA or TBPI (260 μM in PBS) alone. After the incubations the cells were washed three times with PBS and in the following again incubated in Ready Mix Medium. The FITC-labelled conjugates as well as the Alexa-Annexin for the detection of phosphatidyl serine as a signal of the cell death were localized with the confocal laser microscopy (CLSM) as described under 1.6.1. All measurements were performed on living cells.

### 1.6.5 Conjugate 16

Human malignant glioma cells (U373 and LN18) were seeded in 25 cm² culture flasks which contain 3 ml RPMI-1640 Ready Mix Medium with L-Glutamin and 10% FBS (fetal bovine serum)-Gold (PAA laboratories, Pasching, Austria) [37° C., 5% CO₂ (vol/vol)]. The medium was left as it is for one day to enable the accumulation of sufficient MMP-2 secreted by the glioma cells. For the activation of the inactive proform of MMP-2 being present in the medium the latter was incubated on the day of the assay with APMA (final APMA-concentration in the medium: 1 mM, with 1% DMSO) (confluency of the cells: 70%).

In a first assay the BIS-TIBA-conjugate 16 was in each case dissolved without MMP-2 inhibitor in the APMA-containing media of 4 flasks (26 and 130 μM) (both cell lines). In a second assay the BIS-TIBA-conjugate 16 was in turn dissolved in the APMA-media of 4 flasks (26 and 130 μM), however now with MMP-2 inhibitor I.

As controls both glioma cell lines were incubated in a one day old medium both with and also without APMA and inhibitor.

To control the viability MTT-salt and Propidium Iodid (PI) were used as described under 1.4. In addition, phosphatidyl serine in the outer membrane leaflet of apoptotic cells were detected with the annexin-V-Alexa™ 568 reagent according to the recommendation of the manufacturer (Roche Molecular Biochemicals, Indianapolis, USA).

For the confocal laser microscopy an inverse LSM510 laser scanning microscope (Carl Zeiss, Jena, Germany) (Objectives: LD Achroplan 40×0.6, Plan Neofluar 20×0.50, 40×0.75) was used [fluorescence excitation at 480 nm (argon-ion laser) and 534 nm (helium-neon laser)]. Superimposed images of FITC- and Alexa-stained cells were produced. All measurements were performed on living, non-fixed cells in triplicates.

### 1.7 Semi-Thin Sections

A part of the cells which were stained for the FACS analysis, was fixed by paraformaldehyl with 2% Agar, dehydrogenized in ethanol, embedded in Lowicryl K4M (Polysciences, Eppelheim, Germany) and according to the information of the manufacturer UV-polymerised at room temperature. Semi-thin sections (about 0.4 μm) were cut and evaluated by means of fluorescence microscopy.

### 2. Results

### 2.1 Synthesis of the Conjugates

### 2.1.1 Conjugate 1 to 8:

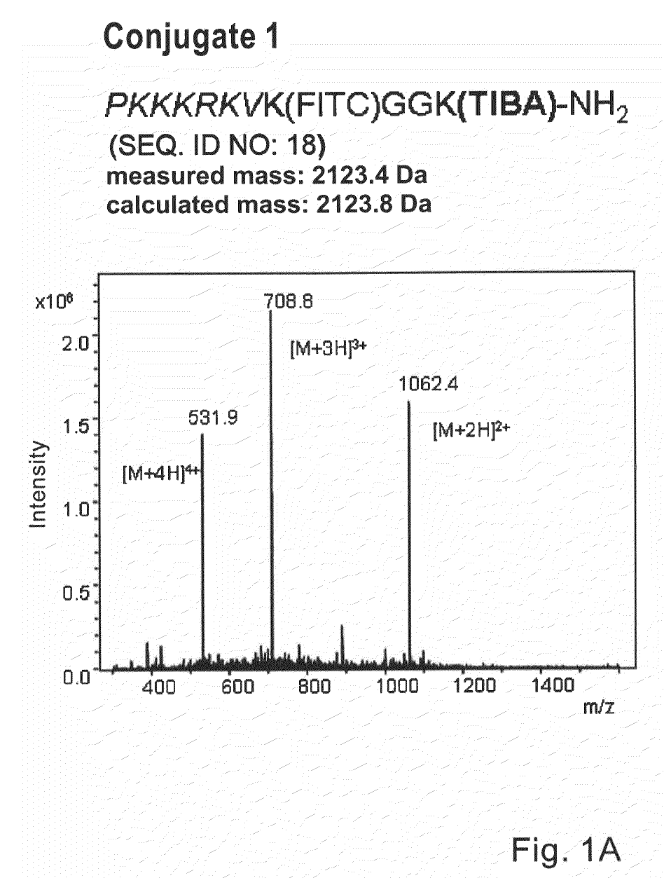

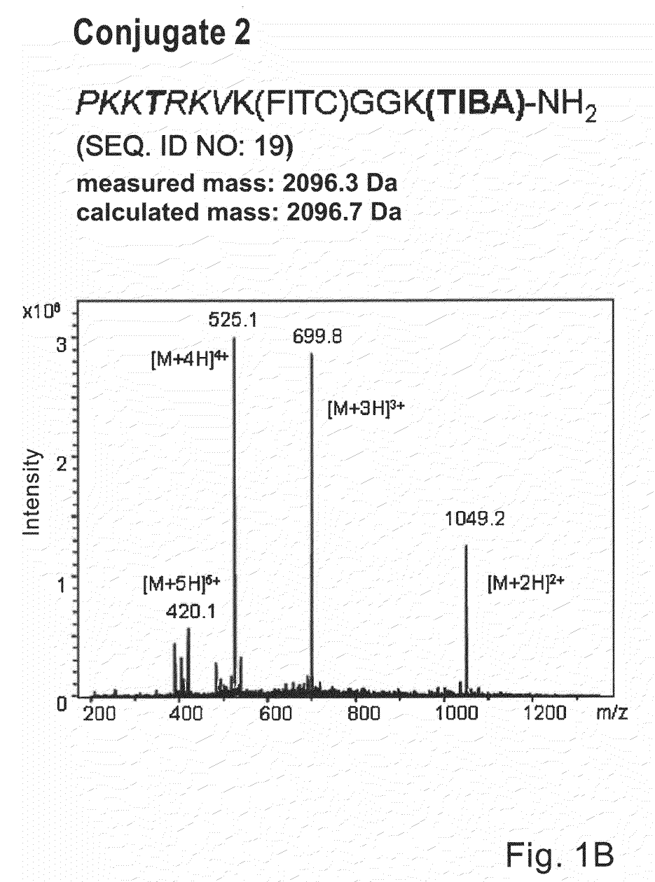

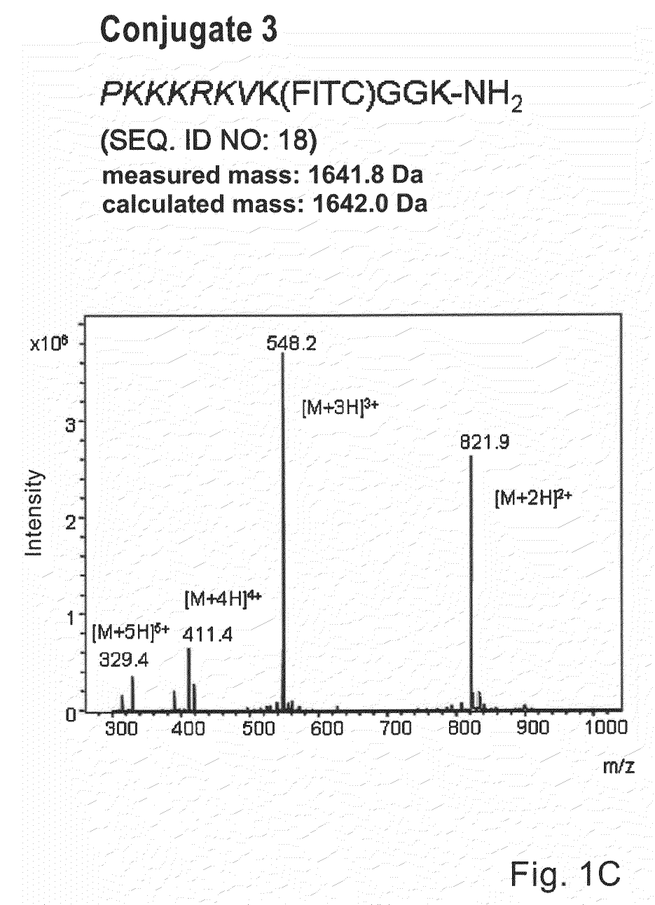

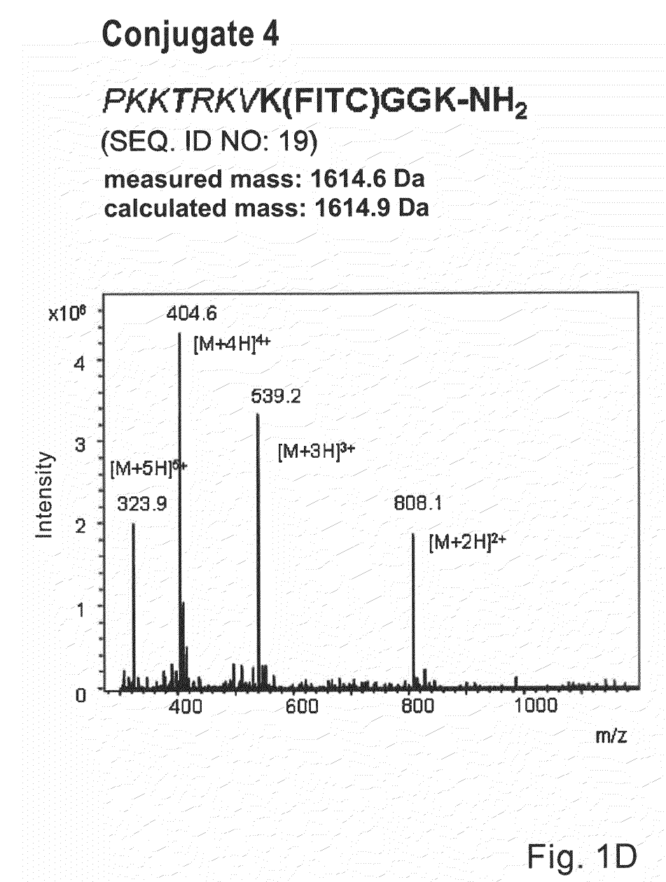

FITC-labelled conjugates were synthesized: The correct NLS of the SV 40 T antigen with TIBA (conjugate 1, K1), a mutant NLS of the SV 40 T antigen with TIBA (conjugate 2, K2), and both of these conjugates without TIBA (conjugates 3 and 4, K3 and K4). The same conjugates however without FITC are designated as conjugates 5 to 8, K5 to K8; table 1, as usual for all conjugates the C-terminus is located on the left side and the N-terminus on the right side, FIG. 1.

The conjugate K1 comprises a molecular weight of 2123.4 Da, the conjugate K2 of 2096.3 Da, the conjugate K3 of 1641.8 Da, the conjugate K4 of 1614.6 Da, the conjugate K5 of 1735.4 Da, the conjugate K6 of 1708.3 Da, the conjugate K7 of 1493.1 Da, the conjugate K8 of 1466.0 Da.

### 2.1.2 Conjugates 9 to 11:

Three FITC-labelled conjugates were synthesized: The NLS of the SV 40 T antigen with the non-iodized benzoic acid (BA) (conjugate 9), the 4-monoiodobenzoic acid (MIBA) (conjugate 10) or the 2,5-diiodobenzoic acid (DIBA) (conjugate 11); table 1.

The conjugate 9 comprises a molecular weight of 1745.95 Da, the conjugate 10 of 1871.83 Da and the conjugate 11 of 1997.72 Da.

### 2.1.3 Conjugate 12:

A FITC-labelled conjugate was synthesized where triiodobenzoic acid (TIBA) was coupled to the prolin; table 1.

The conjugate 12 comprises a molecular weight of 2123.4 Da.

### 2.1.4 Conjugates 13 to 15:

Three further FITC-labelled conjugates were synthesized: The NLS of the SV 40 T antigen with trifluorobenzoic acid (TFBA) (conjugate 13), trichlorobenzoic acid (TCBA) (conjugate 14), and tribromobenzoic acid (TBPI) (conjugate 15); table 1.

The conjugate 13 comprises a molecular weight of 1799.90 Da, conjugate 14 of 1847.81 Da and conjugate 15 of 1997.75 Da.

### 2.1.5 Conjugate 16:

An SV 40 T antigen NLS conjugate labelled with FITC-colorant was constructed which contained two TIBA. These both TIBA are coupled to each other by a cleavable peptide bridge; table 1.

The conjugate 16 comprises a molecular weight of 3255.76 Da.

2.2 Uptake of the Conjugates into the Cell or the Nucleus, Respectively, and Induction of the Apoptosis

### 2.2.1 Conjugates 1 to 8:

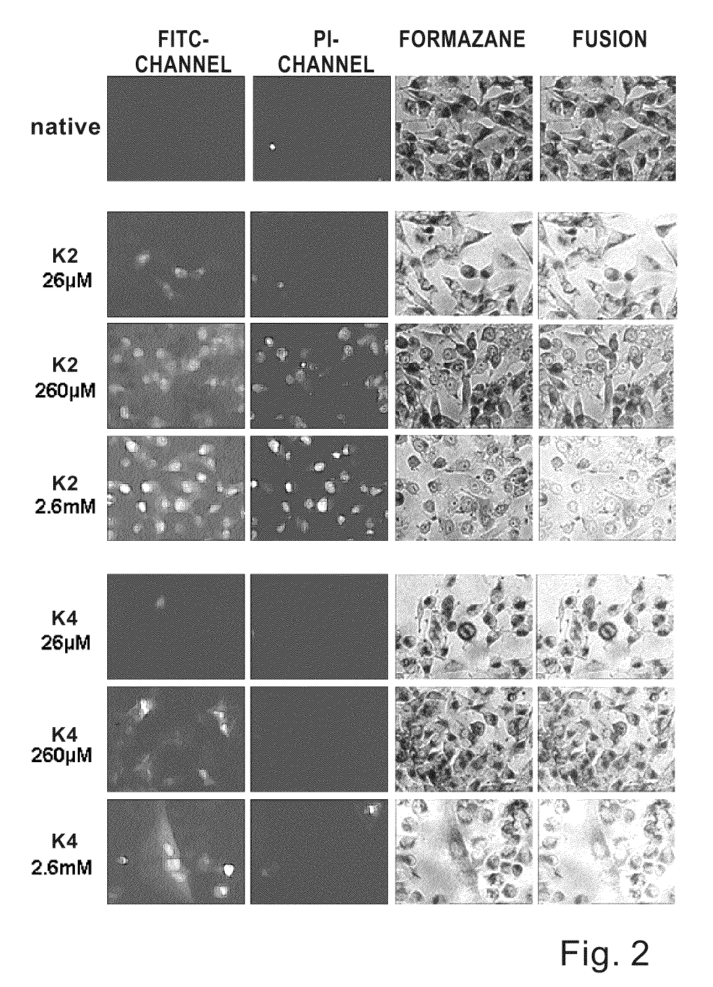

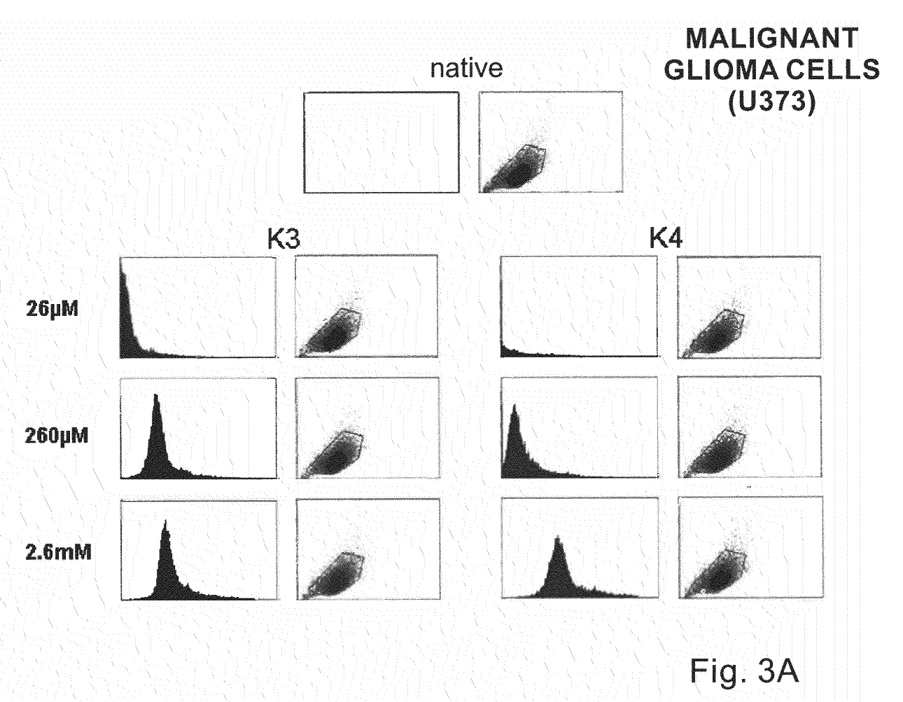

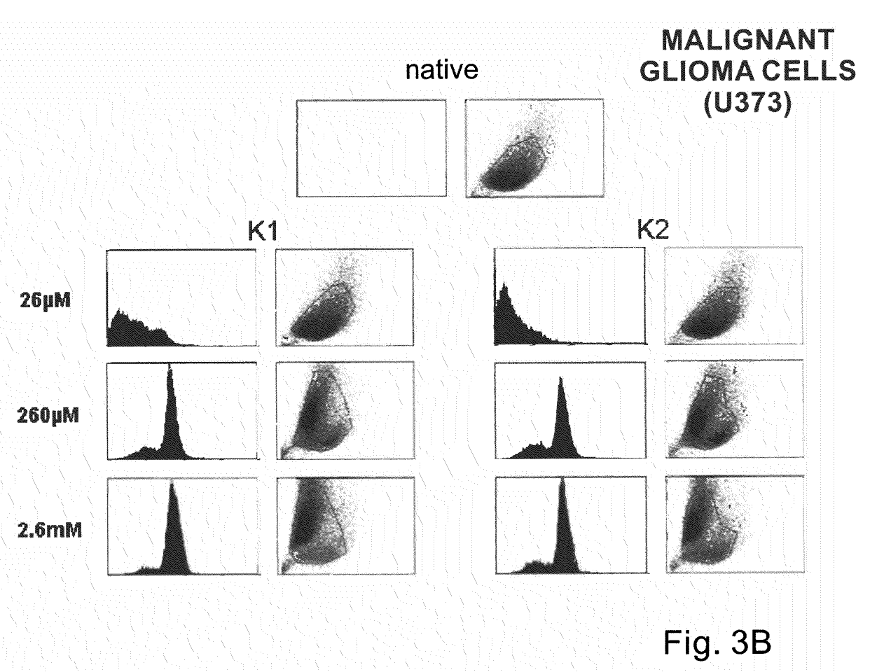

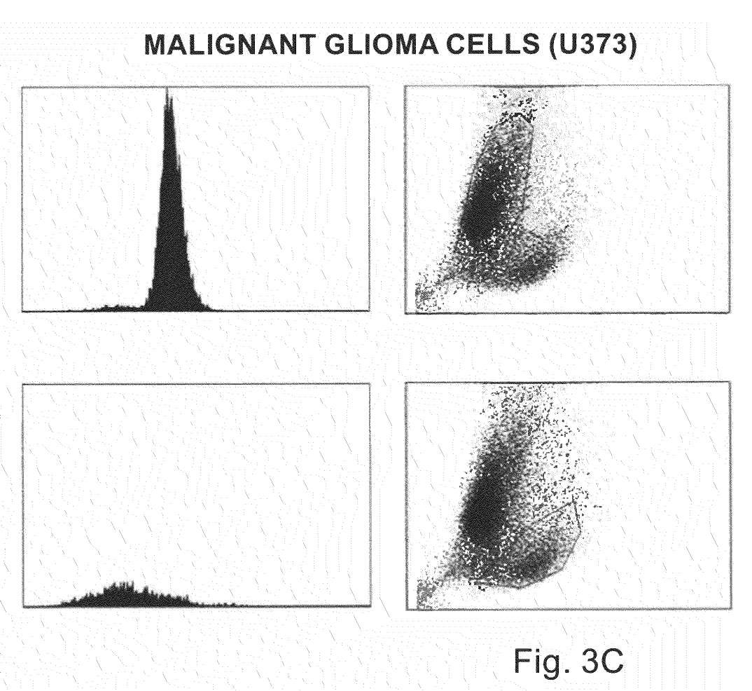

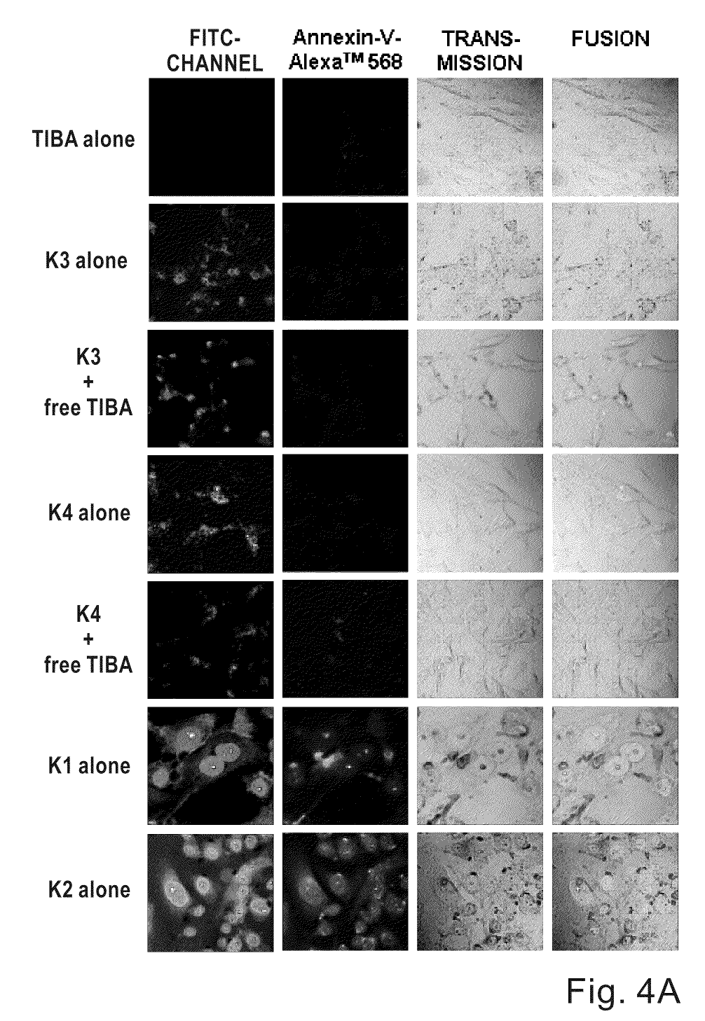

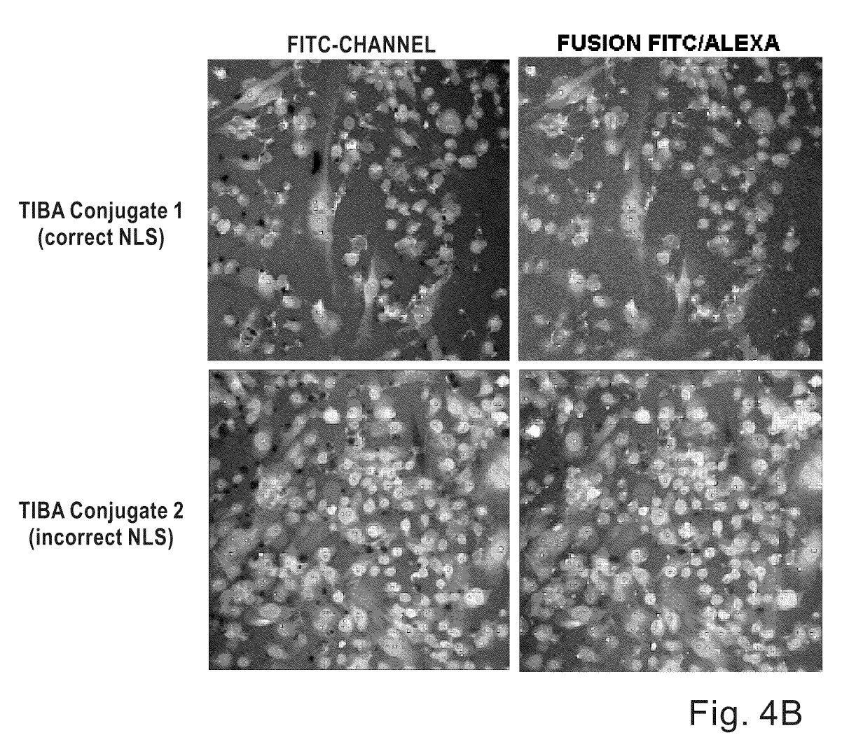

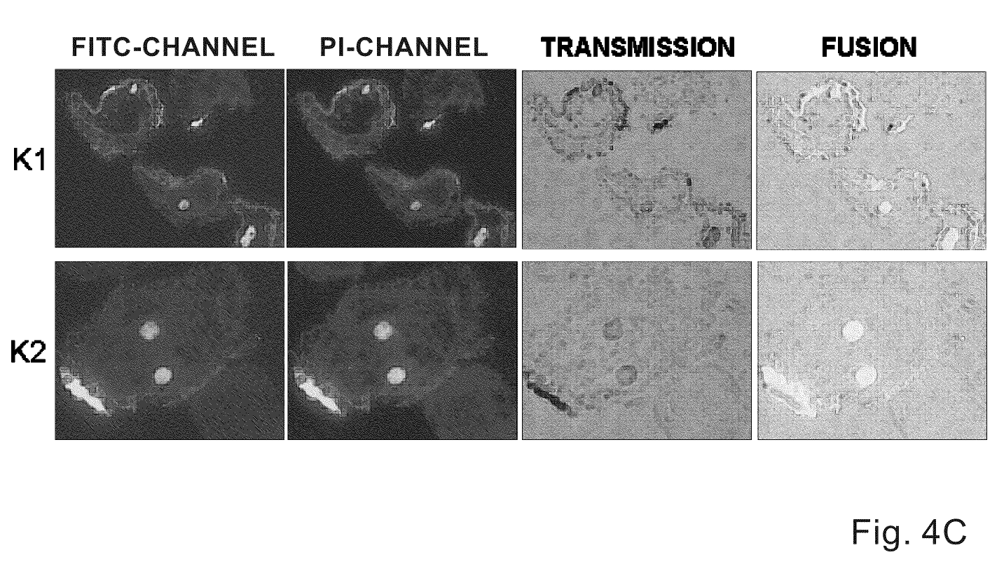

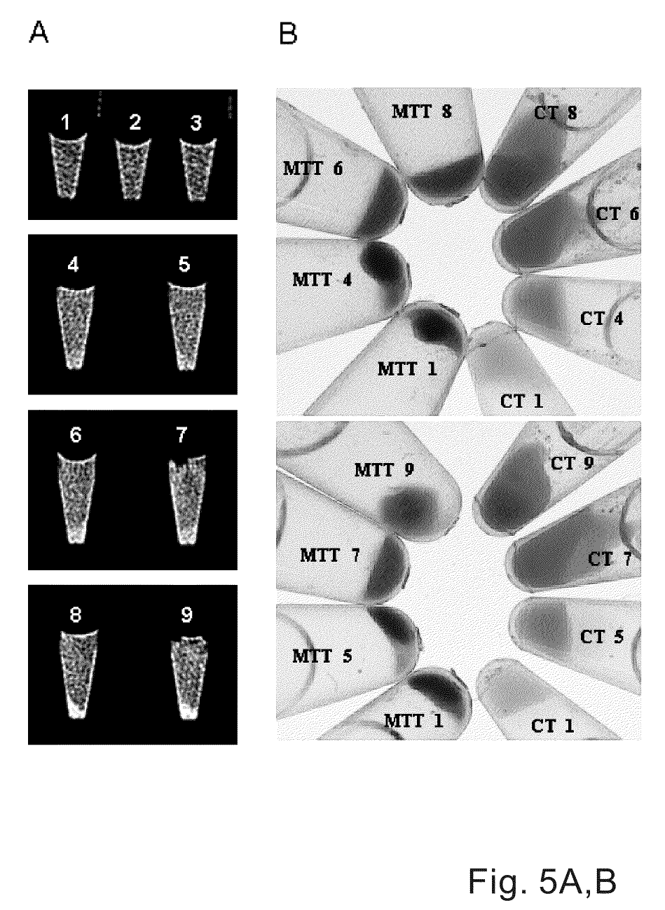

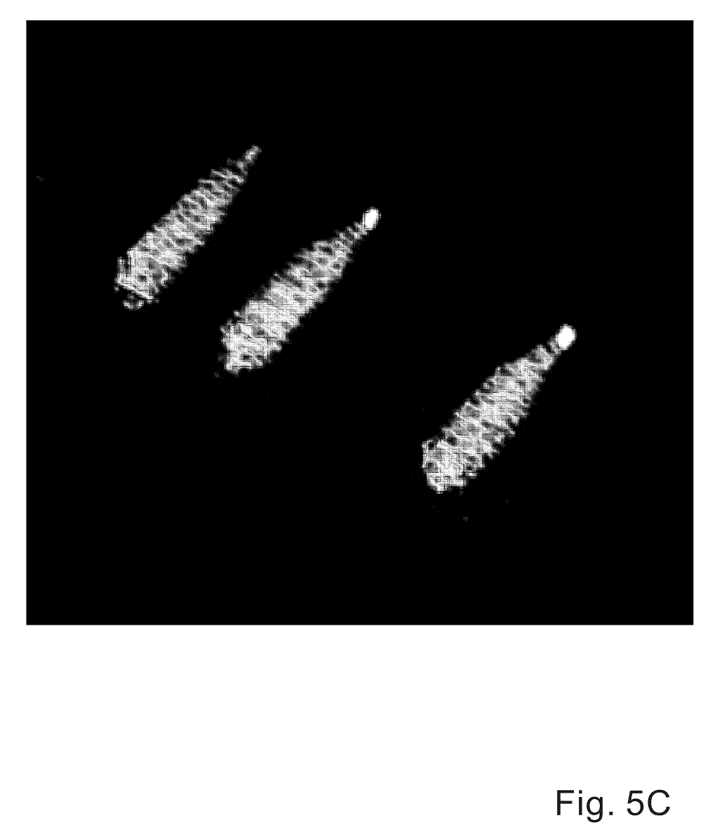

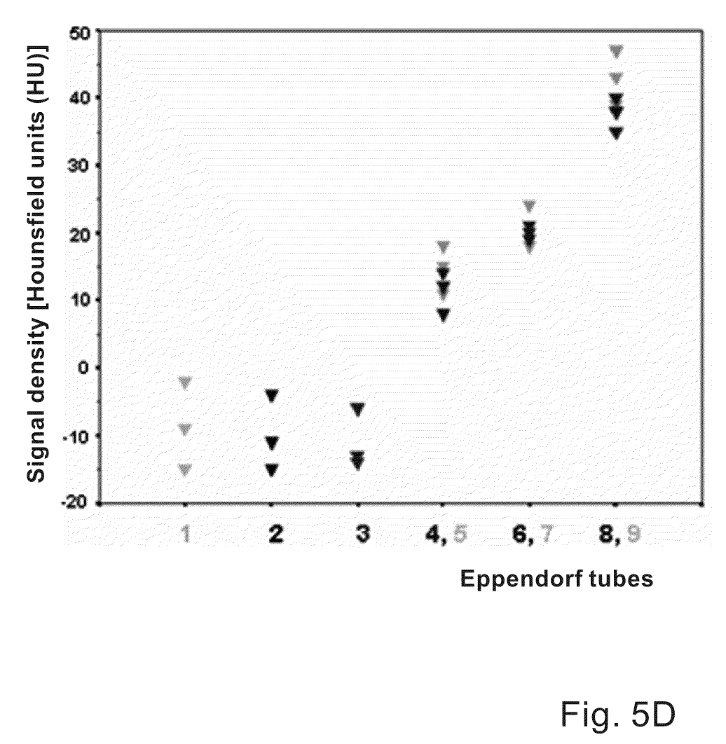

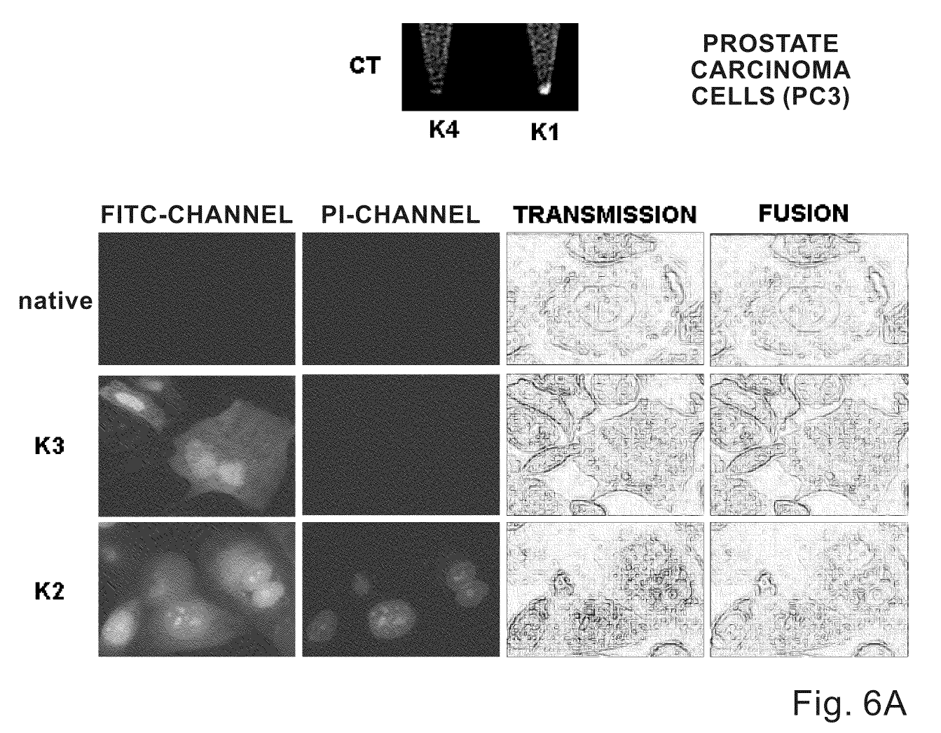

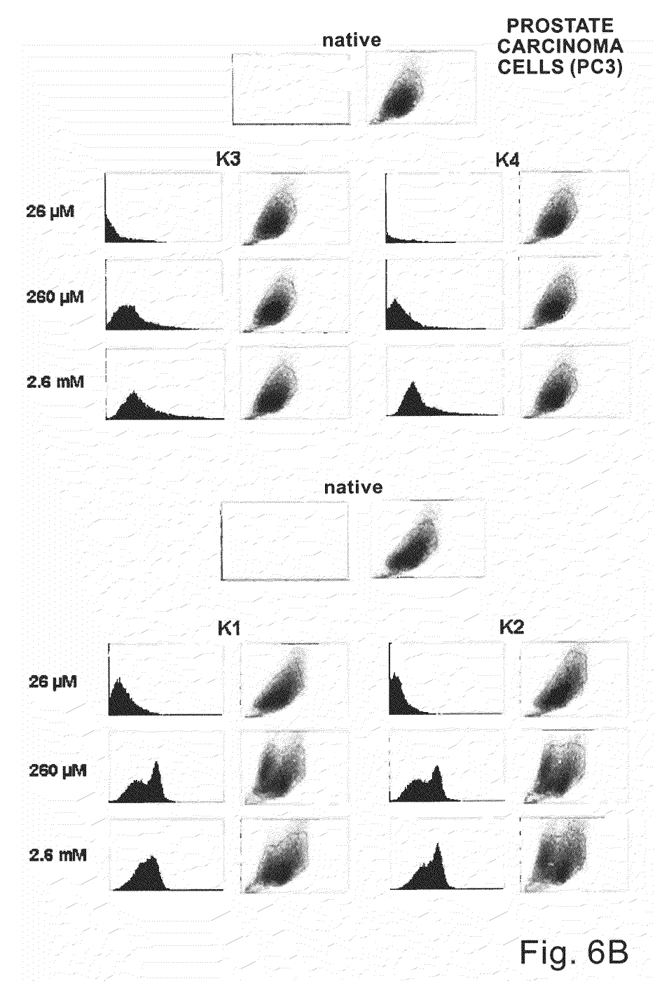

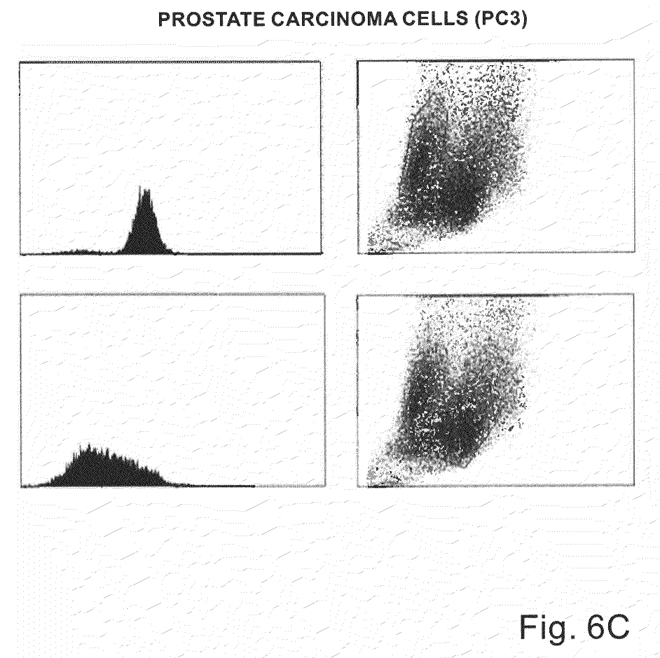

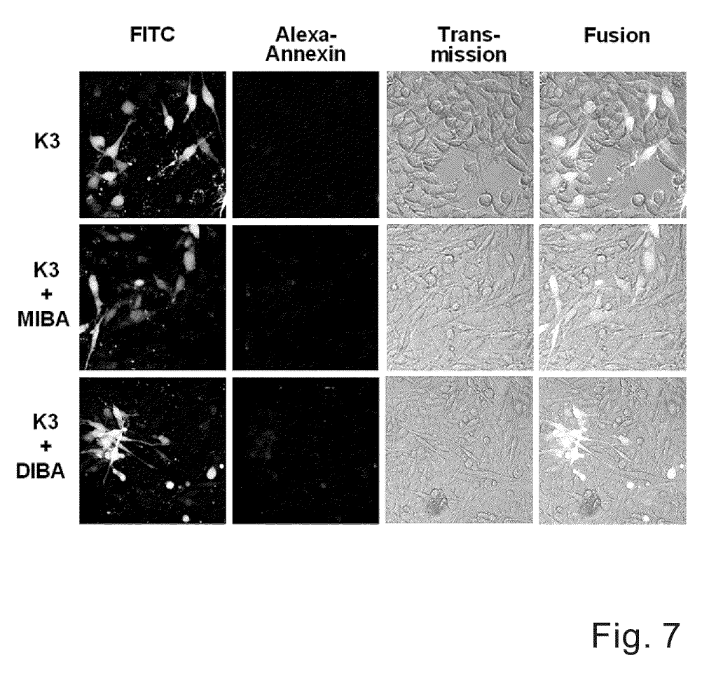

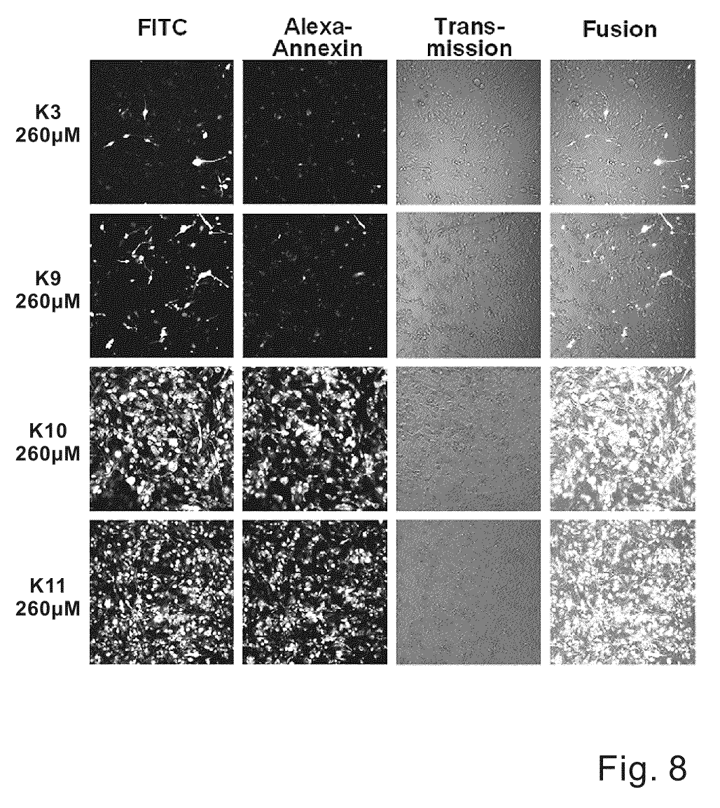

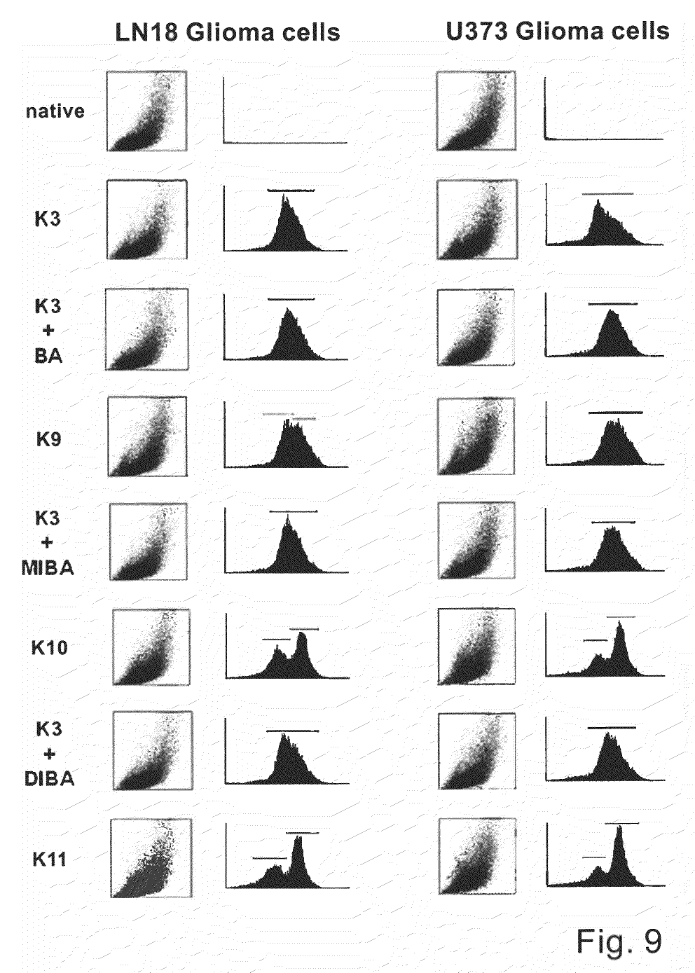

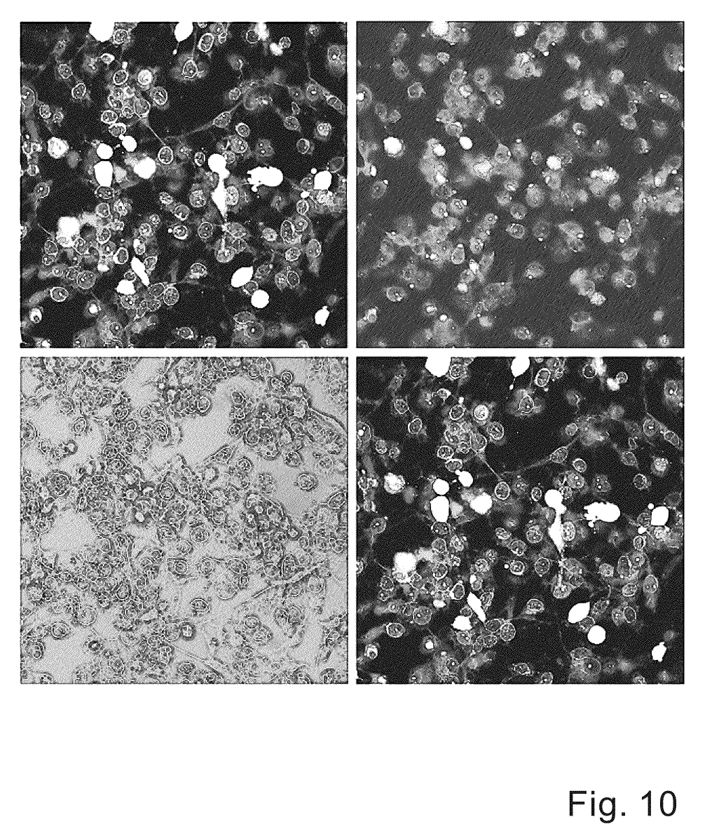

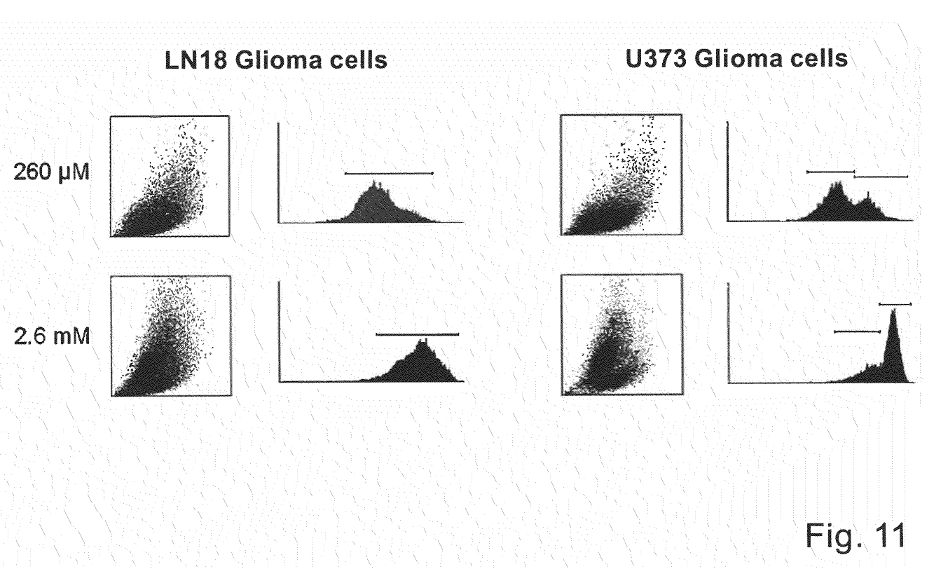

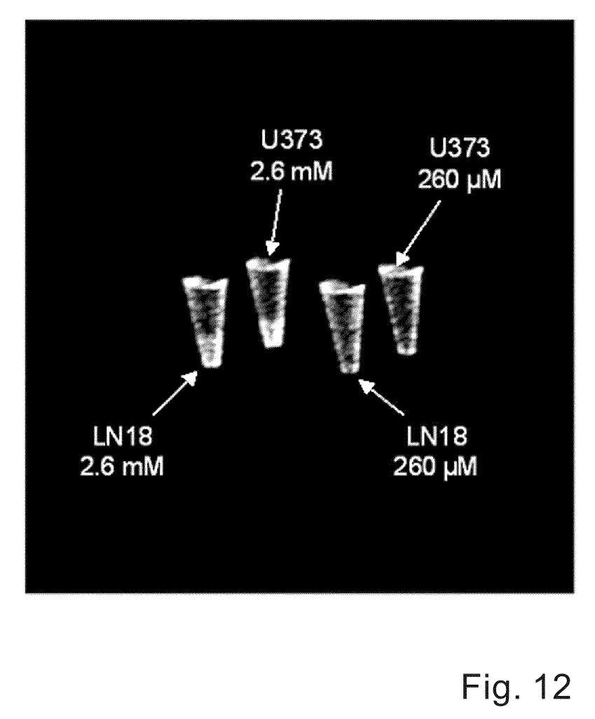

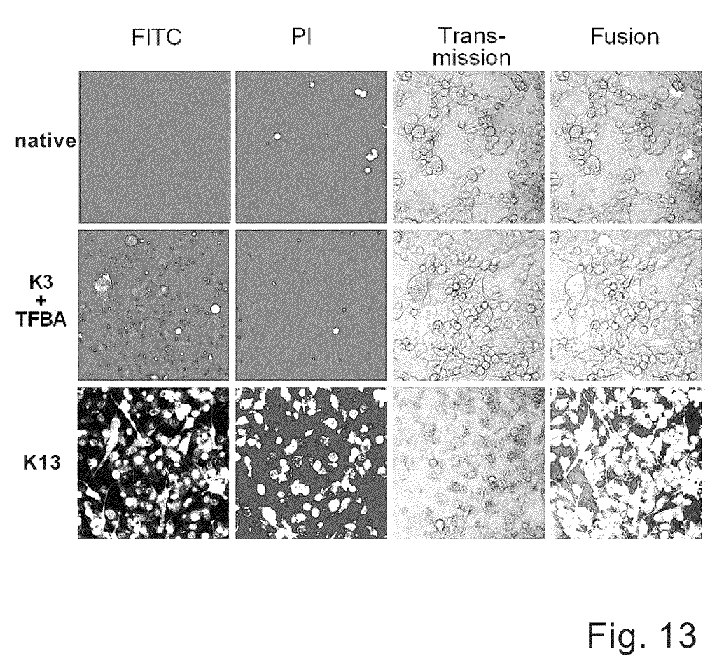

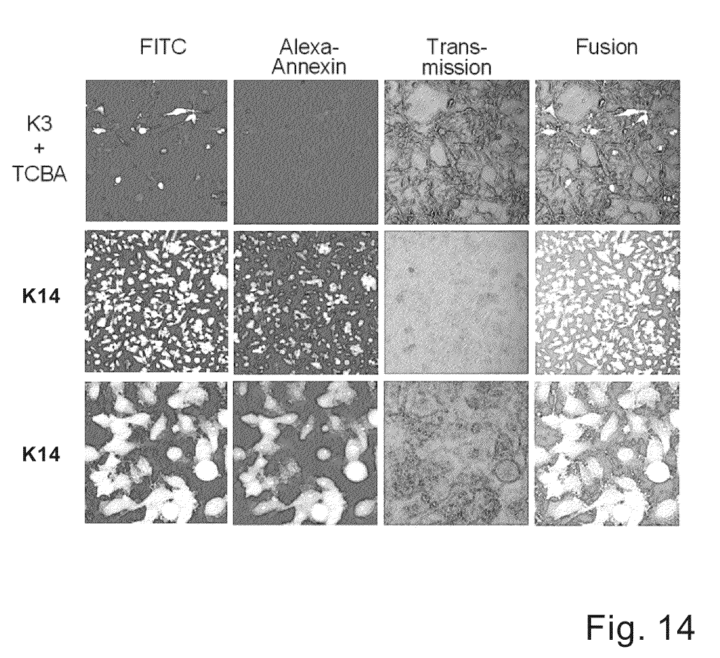

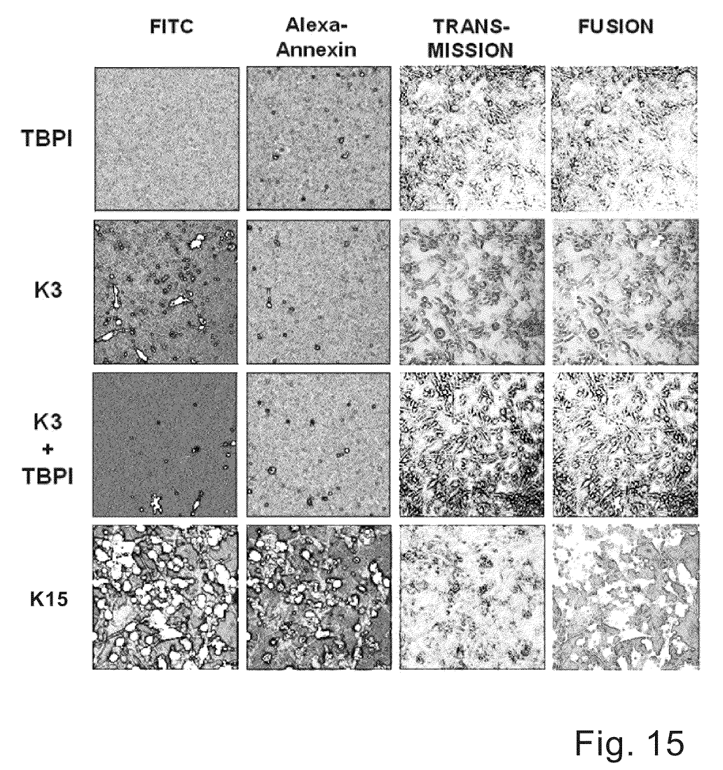

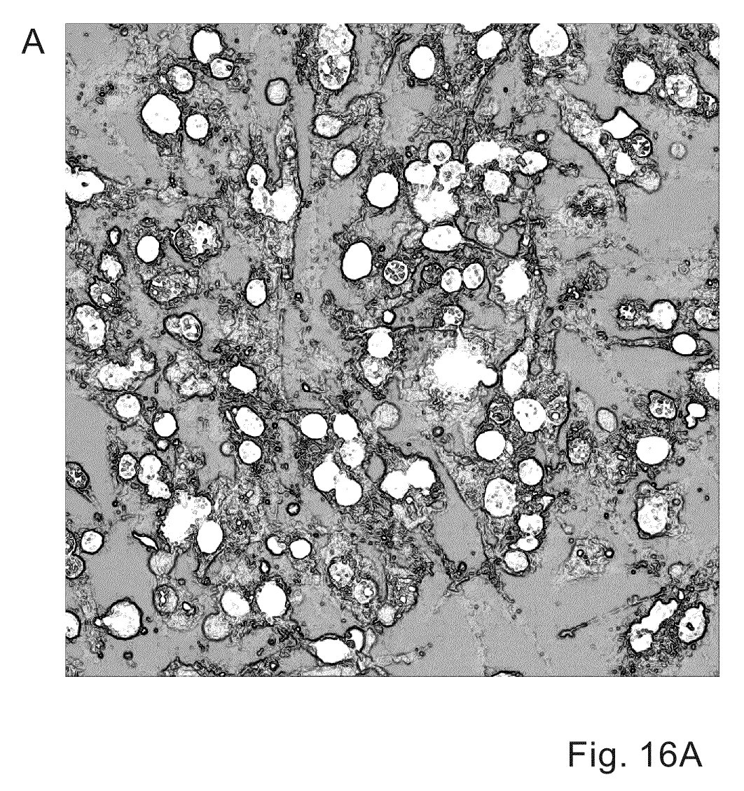

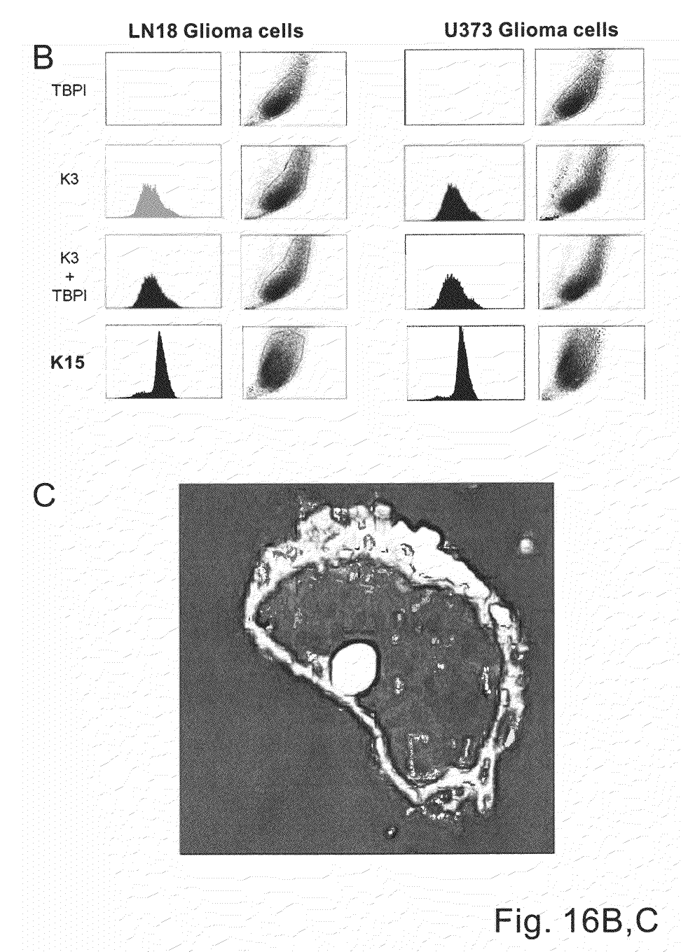

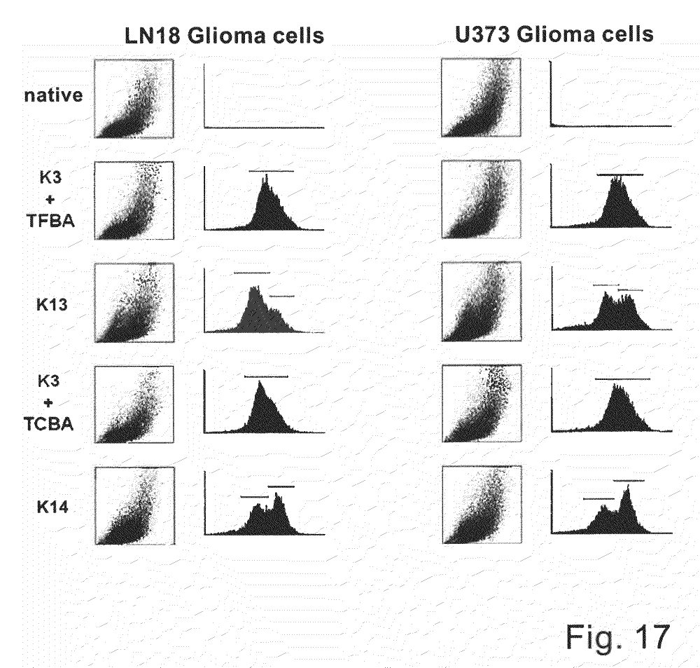

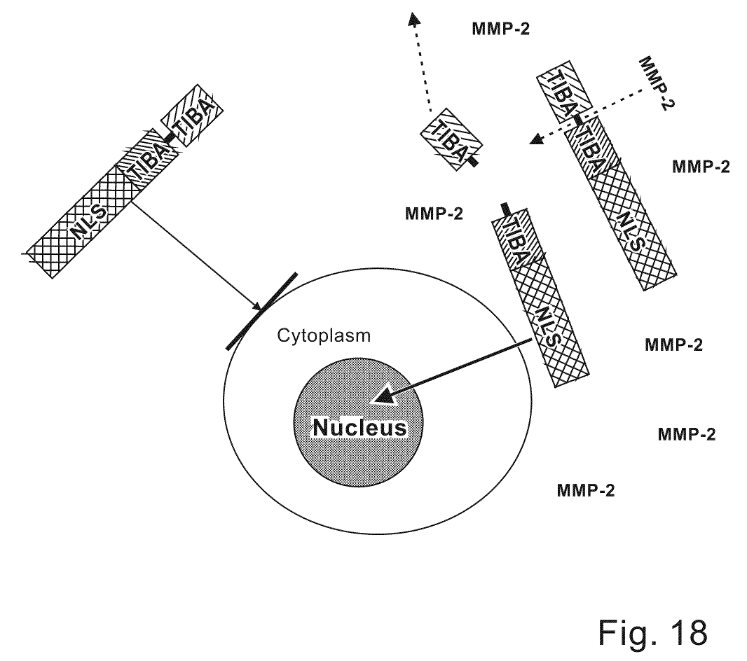

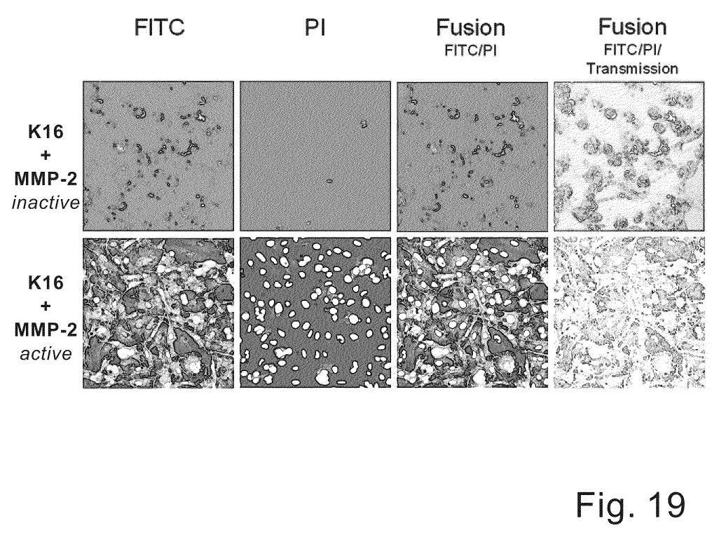

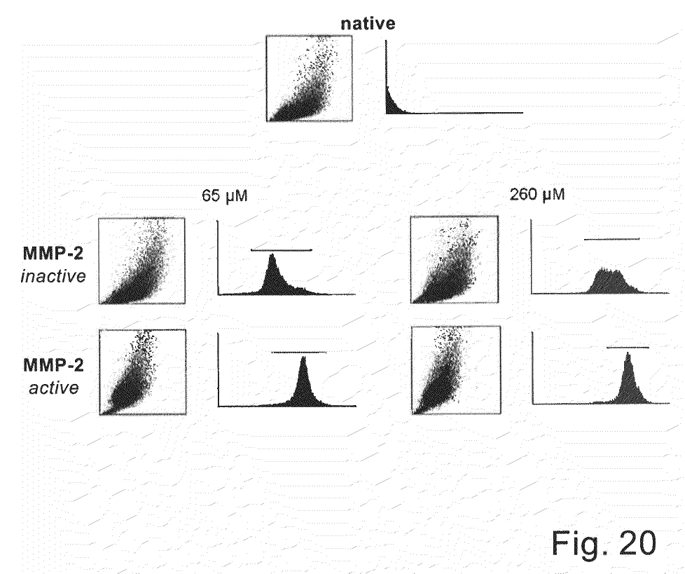

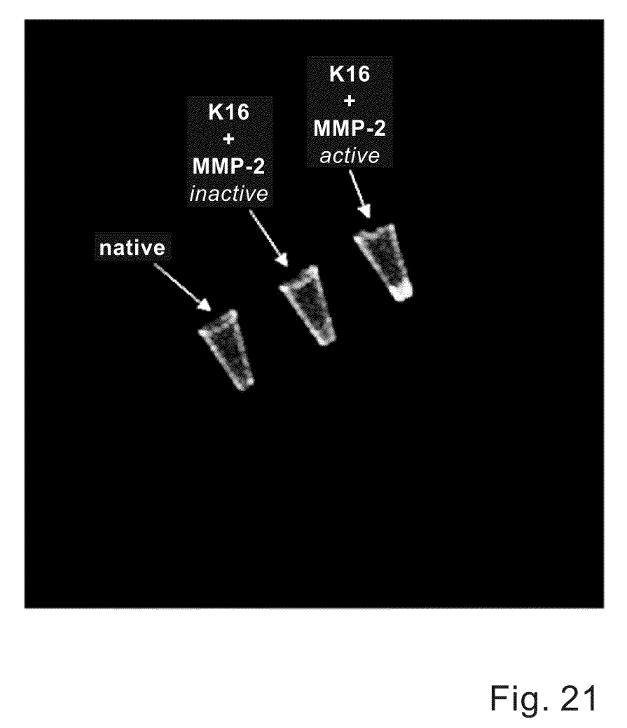

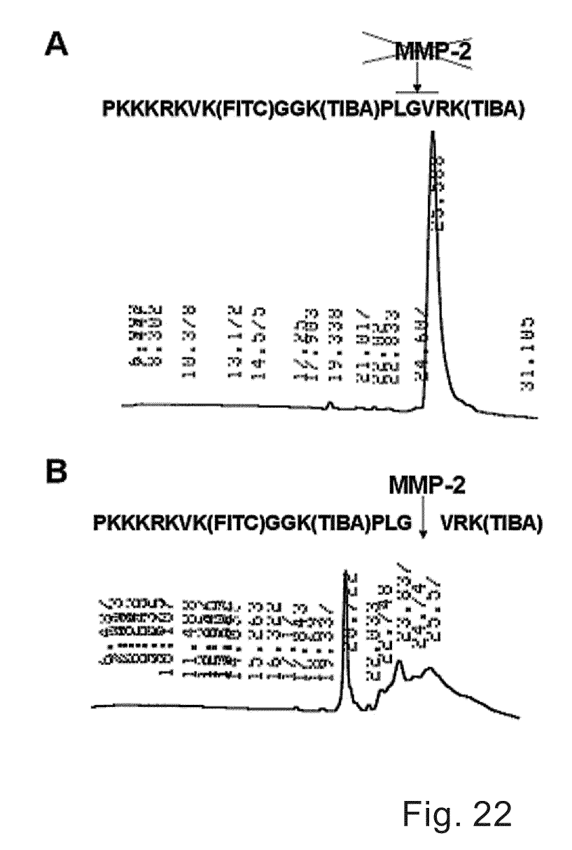

A significant autofluorescence of human malignant U373 glioma cells was excluded prior to the evaluation of the conjugate by fluorescence microscopy (FIG. 2A).

After the incubation with the TIBA conjugates 1 or 2 at a concentration of 26 μM, only a small percentage of the cells (up to 22%) exhibited cytoplasmatic and nuclear staining, as demonstrated by fluorescence microscopy, confocal laser scanning microscopy and fluorescence activated cell sorting (FACS) analysis (FIGS. 2 and 3). These cells showed no signs of cell death, as demonstrated by the production of formazan in the MTT-test and the lack of propidium iodide (PI) uptake (FIGS. 2 and 4a). Side scatter versus forward scatter FACS analysis revealed no changes in the cellular morphology compared to the controls (cells incubated only with PBS) (FIG. 3).

A very marked increase in the proportion of heavily stained cells to 93% was observed after the incubation with the TIBA conjugates at concentrations of 260 μM and 2.6 mM (FIGS. 2, 3, 4). This was associated with a 90% cell death rate (binding of annexin-V-Alexa™ 568 reagent to phosphatidyl serine in the outer membrane leaflet, PI uptake and lack of formazan production in the MTT-test) (FIGS. 2, 4 and 5B). After the incubation with the TIBA conjugates at these higher concentrations, two morphologically distinct cell populations could be distinguished by their forward and side light scatter characteristics (FIG. 3).

No cell death, but only a small number of stained cells (up to 13%) was observed after the incubation with the conjugate that lacked TIBA (conjugates 3 and 4, Table 1) at the same concentrations (26 μM, 260 μM and 2.6 mM) (FIGS. 2, 3 and 4A).

The co-incubation of TIBA (260 μM) together with either of the non-TIBA-containing conjugates 3 and 4 did not result in any changes with respect to the number of stained cells or cell viability. The incubation with TIBA alone did not appear to produce any cytotoxic effects at a concentration of 260 μM (FIG. 4A).

The intracellular staining (especially nucleoli) was confirmed by the examination of semi-thin sections (about 0.4 μm) of the incubated cells (FIG. 4C).

In the CT, the incubation of the cells with PBS alone and with the TIBA conjugates 1 and 2 at varying concentrations (26 μM, 260 μM and 2.6 mM) did result in differences in signal densities (FIGS. 5A and D), although no substantial differences between conjugates 1 and 2 were seen (FIGS. 5A, C and D). The incubation with 260 μM of Ultravist (Iopromid), the contrast agent commonly used in routine clinical investigations, and with 260 μM of TIBA alone did not result in any increase in signal density of the cell pellets compared to the native control (PBS alone) (FIGS. 5A and D).

### 2.2.2 Conjugates 9 to 11:

The human malignant LN18 and U373 glioma cells show after the incubation with PBS buffer alone no autofluorescence in the confocal laser microscopy.

The incubation with benzoic acid, 4-monoiodo benzoic acid, or 2.5-diiodo benzoic acid alone did not result in cytotoxic effects (at a concentration of 260 μM).

After the incubation of the cells with the conjugate 3 which did not contain BA, MIBA or DIBA, only few cells were stained and did not show any signs of cell death (U373 glioma cells: 8%, LN18 glioma cells: 9%) (FIG. 7).

The co-incubation either of BA, MIBA or of DIBA (260 μM) with conjugate 3 which did not contain any of these three components, did not result to a considerable change in the amount of heavily stained cells and of cell viability (FIG. 7).

Also conjugate 9 (conjugate 3 coupled to BA) did only stain a few number of U373 glioma cells (8%) and LN18 glioma cells (9%) (comparable to conjugate 3) (FIG. 8). These few stained cells showed no signs of cell death (no binding of annexin-V-Alexa™ 568 reagent to phosphatidyl serine in the outer membrane leaflet) (FIG. 8).

After the incubation with the MIBA-containing NLS conjugate 10, a strong increase of heavily stained cells (up to 70%) could be observed (FIG. 8). This increase of heavily stained cells was connected with a high cell death rate (annexin-V-Alexa™ 568 staining) (FIG. 8).

A further iodine atom within the benzene ring (conjugate 11) did not result in a further increase of the number of heavily stained cells in comparison to conjugate 10 with only one iodine atom (FIG. 8).

The results of the confocal laser microscopy are reflected in the FACS (fluorescence activated cell sorting) analysis (FIG. 9).

By means of semi-thin sections (about 0.4 μm), it could be demonstrated that the conjugates accumulate in the nucleoli.

### 2.2.3 Conjugate 12:

The human malignant LN18 and U373 glioma cells showed after the incubation with PBS buffer no autofluorescence in the confocal laser microscopy.

However, after the incubation of the cells with the conjugate 12, a large amount (up to 70%) was heavily stained in the nucleus and was then also stained by the annexin-V-Alexa™ 568 reagent (FIG. 10). This means that the cells induced the cell death (FIG. 10). On the semi-thin sections (about 0.4 μm), the conjugate 12 was concentrated in the nucleoli.

In the FACS (fluorescence activated cell sorting) analysis, comparably with the conjugate 1, two morphologically different, viable and non-viable cell populations could be demonstrated (FIG. 11).

In the computer tomography, both cell lines showed only after the incubation at 2.6 mM a clear increase of the signal density in relation to the untreated cells, whereas the U373 in comparison to the LN18 glioma cells showed a slightly higher signal density (FIG. 12). After the incubation of the cells with 260 μM, due to the low signal density, the cells could not be delimited in the Eppendorf tubes, as this applies for the untreated control (FIG. 12).

### 2.2.4 Conjugates 13 to 15

The human malignant LN18 and U373 glioma cells showed after the incubation in pure PBS buffer no autofluorescence in the confocal laser microscopy (FIG. 13). The incubation with trifluorobenzoic acid, with trichlorobenzoic acid or the tribromophenylisocyanate alone (each 250 μM) did not result in any influence on the cell viability (FIG. 15). After the incubation of the cells with the trichlorobenzoic acid (TCBA)-, trifluorobenzoic acid (TFBA)- and the tribromophenylisocyanate (TPBI)-free TITC labelled NLS conjugate 3 only few cells without a sign of cell dead were stained (U373 glioma cell: 8%. LN18 glioma cell: 9%) (FIG. 15). After the incubation with the NLS peptide coupled to benzoic acid (conjugate 9) the staining rate did not increase in comparison to conjugate 3 without benzoic acid; the cells remain viable (FIG. 8).

The coincubation either of free, unbound trichlorobenzoic acid (TCBA), trifluorobenzoic acid (TFBA) or the tribromophenylisocyanate (TPBI) (260 μM) with conjugate 3, which did not contain any of these three components, did not result in any considerable change of the amount of heavily stained cells or the cell viability (FIG. 13 to 15, 16, bottom and 17).

A considerable increase of the heavily stained cells (78%) was observed after the incubation with the TCBA-, TFBA- or the TPBI-containing conjugates 13, 14 or 15 (FIG. 13-16). The heavily stained cells in each case showed signs of cell death (binding of annexin V Alexa™ 568 reagent to phosphatidyl serine of the outer membrane leaflet) (FIG. 13-16). In the computer tomography the TCBA-, TFBA and TPBI-conjugates (260 μM) did not result in an increase of the signal density of the cell centrifugates. In semi thin sections (about 0.4 μm) it could be demonstrated that the conjugates accumulate in the nucleoli (FIG. 16 bottom).

### 2.2.5 Conjugate 16

### Mode of Action:

In FIG. 18 the mode of action of the conjugate 16 is schematically explained on the basis of a specific embodiment. Via a C-terminally located non-shown lysine moiety the first compound in the form of triiodobenzoic acid (TIBA) is covalently bound to the NLS. The second peptide which comprises a cleavage side which is recognized at least by the tumor specific protease MMP-2, is bound via a peptide bond to the carboxy group of the lysine moiety. The second peptide is shown as a thin bar. The C-terminus of the second peptide is followed by the second compound. This is, in the form of triiodobenzoic acid (TIBA), also bound to the second peptide via a non-shown lysine residue which is C-terminally located in the second peptide.

This conjugate 16 according to the invention cannot enter healthy-non-transformed cells due to its size and the lack of MMP-2 (left). Only in the presence of transformed tumor cells which secrete MMP-2 into their neighborhood, the second peptide is cleaved off. The released remaining conjugate 16 can enter the cytoplasm and the nucleus of the tumor cell due to its reduced size (right).

After the induction of the apoptosis in the tumor cells by the remainder conjugate 9 the cleavage products are eliminated by macrophages and maybe excreted from the organism.

### Tumor Cell Specificity:

The human malignant LN18 and U373 glioma cells (adherent and detached) do not show a cell fluorescence in the confocal laser microscopy (CLSM) after incubation with APMA (4-amiphenyl mercuric acetate) containing RPMI medium either with or without inhibitor.

Both the inhibitor and also APMA in the medium did not influence the cell viability. In the presence of the inhibitor in APMA containing medium the incubation of adherent and attached LN18 and U373 glioma cells with the BIS-TIBA conjugate 16 (130 μM) did only result in few stained nuclei in the confocal laser microscopy (CSLM) (FIG. 19) and FACS (fluorescence activated cell sorting) analysis (FIG. 20). These cells remain viable.

In the absence of the inhibitor of the APMA containing medium the incubation of adherent and detached LN18 glioma cells with the BIS-TIBA conjugate 16 (130 μM) results in a strong increase of the staining of the nuclei in the CLSM (FIG. 19) and the FACS analysis (FIG. 20) whereas these cells were necrotic [uptake of propidium iodide (PI) in the nucleus] (FIG. 19).

In the computer tomography the cells showed after the incubation with inhibitor containing medium only a small increase of the signal density in comparison with the untreated control (13 and 130 μM) (FIG. 21). A heavy increase of the signal density could be observed after the incubation of the cell with the BIS-TIBA conjugate (K16) (130 μM) in medium without inhibitor (FIG. 21).

In the HPLC (high performance liquid chromatography) it could be demonstrated that the BIS-TIBA conjugate (K16) is cleaved in the presence of the active MMP-2 but also of MPP-2 proform activated by APMA, and that this cleavage could be prevented in the presence of the inhibitor (FIG. 22).

### 3. Summary

The inventors provide a conjugate which is an improved contrast medium and also an apoptosis inducing therapeutic agent. According to a preferred embodiment the conjugate is tumor specific and enables a targeted diagnosis and/or therapy of a tumor disease.

TABLE 1

| | | |
|---|---|---|
| K1 | PKKKRKVK(FITC)GGK(TIBA) | (SEQ. ID NO: 18) |
| K2 | PKK [symbol] RKVK(FITC)GGK(TIBA) | (SEQ. ID NO: 19) |
| K3 | PKKKRKVK(FITC)GGK | (SEQ. ID NO: 18) |
| K4 | PKK [symbol] RKVK(FITC)GGK | (SEQ. ID NO: 19) |
| K5 | PKKKRKVKGGK(TIBA) | (SEQ. ID NO: 18) |
| K6 | PKK [symbol] RKVKGGK(TIBA) | (SEQ. ID NO: 19) |
| K7 | PKKKRKVK(TIBA) | (SEQ. ID NO: 14) |
| K8 | PKK [symbol] RKVK(TIBA) | (SEQ. ID NO: 15) |
| K9 | PKKKRKVK(FITC)GGK(BA) | (SEQ. ID NO: 18) |
| K10 | PKKKRKVK(FITC)GGK(MIBA) | (SEQ. ID NO: 18) |
| K11 | PKKKRKVK(FITC)GGK(DIBA) | (SEQ. ID NO: 18) |
| K12 | P(TIBA)KKKRKVK(FITC)GGK | (SEQ. ID NO: 18) |
| K13 | PKKKRKVK(FITC)GGK(TFBA) | (SEQ. ID NO: 18) |
| K14 | PKKKRKVK(FITC)GGK(TCBA) | (SEQ. ID NO: 18) |
| K15 | PKKKRKVK(FITC)GGK(TBPI) | (SEQ. ID NO: 18) |
| K16 | PKKKRKVK(FITC)GGK(TIBA)PLGVRK(TIBA) | (SEQ. ID NO: 20) |

1. Conjugate, comprising
a first compound comprising the following formula: 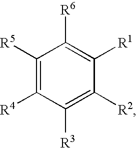
wherein R¹, R², R³, R⁴, and R⁵, independently from each other, each correspond to a halogen or a hydrogen, and wherein R⁶ corresponds either to a carboxyl group (COOH) or to isothiocyanate (S═C═N), and
at least one peptide,
wherein the conjugate is configured in such a manner that it can penetrate the cell membrane and the nuclear membrane.

2. Conjugate according to claim 1, wherein the halogen of the first compound is selected from the group consisting of: iodine, bromine, fluorine, chlorine, and astatine.

3. Conjugate according to claim 1, wherein the first compound is selected from the group consisting of: triiodobenzoic acid (TIBA), 5-iodobenzoic acid (5-IBA, 5-MIBA), 4-iodobenzoic acid (4-IBA, 4-MIBA), 2,3-diiodobenzoic acid (2,3-DIBA), 3,5-diiodobenzoic acid (3,5-DIBA), 2,5-diiodobenzoic acid (2,5-DIBA), trichlorobenzoic acid (TCBA), trifluorobenzoic acid (TFBA), tribromobenzoic acid (TBBA), tribromphenyl isothiocyanate (TBPI).

4. Conjugate according to claim 1, wherein the first peptide comprises a positive net charge.

5. Conjugate according to claim 1, wherein the first peptide comprises 2 to 20 amino acids.

6. Conjugate according to claim 1, wherein the first peptide comprises 7 amino acids.

7. Conjugate according to claim 1, wherein the first peptide is derived from a nuclear localization sequence (NLS).

8. Conjugate according to claim 1, wherein the first compound is, via its carboxyl group, bound to the first peptide.

9. Conjugate according to claim 1, wherein the first peptide comprises a free amino function of a side chain via which it is bound to the first compound.

10. Conjugate according to claim 9, wherein the first peptide comprises a C-terminally located lysine moiety comprising an ε-amino function, via which the first peptide is bound to the first compound.

11. Conjugate according to claim 1, wherein the first peptide comprises the amino acid sequences PKKKRKV (SEQ ID NO: 1) or PKKTRKV (SEQ ID NO: 2).

12. Conjugate according to claim 1 further comprising a detectable marker.

13. Conjugate according to claim 12, wherein the detectable marker comprises a lysine moiety comprising an ε-amino function, via which it is bound to the first peptide.

14. Conjugate according to claim 12, further comprising an amino acid spacer located between the detectable marker and the first compound.

15. Conjugate according to claim 14, wherein the amino acid spacer comprises two amino acids.

16. Conjugate according to claim 15, wherein the amino acid spacer comprises the amino acid sequence GG (SEQ ID NO: 3).

17. Conjugate according to claim 1, further comprising at least one component conferring upon said conjugate a tumor cell specificity or a specificity for virus infective cells.

18. Conjugate according to claim 17, wherein the component comprises a third peptide which (i) comprises a charge which at least neutralizes the positive net charge of the first peptide, and (ii) is bound to the conjugate via a second peptide which comprises an amino acid recognition sequence for a tumor cell or virus specific enzyme.

19. Conjugate according to claim 17, wherein the component comprises the following, namely
a second compound comprising the following formula: 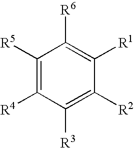
wherein R¹, R², R³, R⁴, and R⁵, independently from each other, each correspond to a halogen or a hydrogen, and wherein R⁶ corresponds either to a carboxyl group (COOH) or to isothiocyanate (S═C═N), and
a second peptide bound to the second component, the second peptide comprising an amino acid recognition sequence for a tumor cell or virus specific enzyme,
wherein the component is bound to the conjugate via the second peptide.

20. Conjugate according to claim 19, wherein the halogen of the first compound is selected from the group consisting of: iodine, bromine, fluorine, chlorine, and astatine.

21. Conjugate according to claim 19, wherein the first compound is selected from the group consisting of: triiodobenzoic acid (TIBA), 5-iodobenzoic acid (5-IBA, 5-MIBA), 4-iodobenzoic acid (4-IBA, 4-MIBA), 2,3-diiodobenzoic acid (2,3-DIBA), 3,5-diiodobenzoic acid (3,5-DIBA), 2,5-diiodobenzoic acid (2,5-DIBA), trichlorobenzoic acid (TCBA), trifluorobenzoic acid (TFBA), tribromobenzoic acid (TBBA), tribromphenyl isothiocyanat (TBPI).

22. Conjugate according to claim 19, wherein the tumor cell or virus specific enzyme is selected from the group consisting of matrixmetalloproteases (MMP), catapsines, prostate specific antigen (PSA), herpes simplex virus protease, human immunodeficience virus protease, cytomegalovirus protease, caspase, interleukin-βconverting enzyme, thrombin.

23. Conjugate according to claim 19, wherein the second peptide comprises an amino acid sequence selected from the group consisting of: PLGLR (SEQ ID NO: 4), PLGVA (SEQ ID NO: 5) and PLGLA (SEQ ID NO: 13).

24. Conjugate according to claim 19, wherein the second peptide comprises a C-terminally located lysine moiety comprising an ε-amino function via which the second peptide is bound to the second compound.

25. Conjugate according to claim 19, wherein it comprises a third peptide comprising a positive net charge.

26. Conjugate according to claim 25, wherein the third peptide comprises 2 to 20 amino acids.

27. Conjugate according to claim 25, wherein the third peptide comprises 7 amino acids.

28. Conjugate according to claim 25, wherein the third peptide is derived from a nuclear localization sequence (NLS).

29. Conjugate according to claim 25, wherein the third peptide comprises a free amino function of a side chain, via which it is bound to the second compound.

30. Conjugate according to claim 25, wherein the third peptide comprises an N-terminally located lysine moiety comprising an ε-amino function, via which the third is bound to the compound.

31. Conjugate according to claim 25, wherein the third peptide comprises the amino acid sequence PKKKRKV (SEQ ID NO: 1) or PKKTRKV (SEQ ID NO: 2).

32. Conjugate according to claim 17, wherein the component comprises a nucleic acid molecule which, under stringent conditions, hybridizes to oncogenes.
